(19) United States
(12) Patent Application Publication
(10) Pub. No.: US 2006/0194332 A1
(43) Pub. Date: Aug. 31, 2006

(54) Gas sensor and method of fabricating a gas sensor

(75) Inventors: Hiroyuki Wado, Toyota-city (JP); Makiko Sugiura, Obu-city (JP); Yukihiro Takeuchi, Nishikamo-gun (JP); Takao Iwaki, Chiryu-city (JP)

Correspondence Address: POSZ LAW GROUP, PLC, 12040 SOUTH LAKES DRIVE, SUITE 101, RESTON, VA, 20191, US

(73) Assignee: DENSO CORPORATION, Kariya-city (JP)

(21) Appl. No.: 11/410,093

(22) Filed: Apr. 25, 2006

Related U.S. Application Data
(62) Division of application No. 10/106,455, filed on Mar. 27, 2002.

(30) Foreign Application Priority Data
Mar. 28, 2001 (JP) 2001-93953
Apr. 5, 2001 (JP) 2001-107758
Apr. 25, 2001 (JP) 2001-128036

(51) Int. Cl. G01N 33/00

(52) U.S. Cl. 436/151

(57) ABSTRACT

A more reliable gas sensor includes a support film formed on a surface of a substrate and a heater electrode. Surrounding the heater electrode is a heater electrical insulation layer 4. Detection electrodes are formed above the electrical insulation layer. A flat insulating layer is formed over the heater insulation layer, and surfaces of the detection electrodes are exposed and flush with the upper surface of the flat insulating layer. A sensitive film is formed above the flat insulating layer in contact with the surfaces of the detection electrodes. A hollow cavity is formed in the substrate.

### CROSS REFERENCES TO RELATED APPLICATIONS

This application relates to and claims priority from Japanese Patent Application No. 2001-93953, filed on Mar. 28, 2001, application No. 2001-107758, filed on Apr. 5, 2001, and application No.2001-128036, filed on Apr. 25, 2001, each of which is incorporated by reference.

### BACKGROUND OF THE INVENTION

The present invention relates to a gas sensor for identifying a gas by using a sensitive film, a physical value of which changes according to the surrounding gas, to a method of fabricating the gas sensor and to a method of detecting a gas.

There are various existing gas sensors formed with a sensitive film, a physical value of which is changed by adsorption, desorption or the like of a gas, on a substrate. The film is capable of calculating a concentration of the gas by measuring the change in the physical value of the sensitive film.

Favorable characteristics of a gas sensor include high sensitivity, excellent selectivity, high response speed, reliability, ease of fabrication, small-size, and low power consumption.

Sensitivity or selectivity of such a gas sensor is significantly dependent on the temperature of the sensitive film, and therefore a heater is provided in the vicinity of the film and the temperature of the film is controlled to a specific temperature (300° C. to 500° C.) by using, for example, a control circuit. Such a gas sensor with a heater layer is disclosed, for example, in JP-A No. 11-201929. FIG. 49 is a sectional view of the gas sensor.

As shown by FIG. 49, a first electrically insulating layer J2 is formed above a substrate J1 of silicon or the like, and a heater layer J3 is formed above the first electrically insulating layer J2. A second electrically insulating layer J4 is formed above the heater layer J3. Above the second electrically insulating layer J4, there are formed an electrode layer J5, which is electrically connected to the heater layer J3, and a temperature sensor J6.

A third electrically insulating layer J7 is formed further above the substrate and a detection electrode J8 is formed above the third electrically insulating layer J7. A sensitive film J9 is formed above the detection electrode J8. Therefore, the detection electrode J8 is arranged below the sensitive film J9, and the temperature sensor J6 and the heater layer J3 are arranged in the second and the third electrically insulating layers J7 and J4.

The temperature of the sensitive film J9 is made uniform by providing the temperature sensor J6 above the heater layer J3 and by executing feedback control. A change in a physical value of the sensitive film J9 at a predetermined temperature is detected by the detection electrode J8 and the kind of gas or the concentration of a gas in an environment is measured.

In such a gas sensor, the second and the third electric insulating layers J4 and J7 are made thin for arranging the heater layer J3 as near as possible to the sensitive film J9, to elevate the temperature of the sensitive film J9 with little power. Therefore, recesses and projections or steps, which are caused by the patterns of the heater layer J3 and the temperature sensor J6, emerge also on the second the third electric insulating layers J4 and J7. As a result, the sensitive film J9 is formed on a face formed with recesses and projections.

Hence, according to JP-A No. 11-201929, a method that uses bias sputter is used to flatly form the third electrically insulating layer J7. However, recesses and projections of the detection electrode J8 remain on the face formed with the sensitive film J9.

In such a gas sensor, the sensitive film J9 is formed by a thin film, and accordingly, when the sensitive film J9 is formed on the face formed with recesses and projections, the sensitive film J9 is subject to breakage. Although the sensitive film J9 is not immediately broken, when the gas sensor is used over a long period of time, a crack may form on a surface of the sensitive film J9 by thermal expansion of the heater layer J3 arranged below the sensitive film J9, and this limits the life of the product.

When the detection electrode J8 and the heater layer J3 are provided below the sensitive film J9, a number of layers are formed such that the electric insulating layers must be provided between the detection electrode J8 and the heater layer J3, and therefore, there are many manufacturing steps.

It is known that a sensitive film is sensitive not only to one kind of a gas but also to a plurality of kinds of gases and when, for example, a sensitive film is intended to detect one kind of gas, the selectivity is low.

With regard to this problem, according to JP-A No. 2-88958, there is a technology for identifying the kind and concentration of a gas from a change in a physical value by measuring the change in the physical value at a plurality of temperatures. That is, the device uses the dependency of the change in the physical value of the sensitive film on the kind of the gas and the temperature.

However, the sensitivity of the sensitive film is reduced when gas or the like is adsorbed in the sensitive film. Therefore, when the amount of adsorbed gas or the like differs in a sensitive film before measuring the gas, the change in the physical value of the sensitive differs even under an equal gas environment, and the gas may be wrongly identified.

Even when the above-described technology is used, the change in the physical value of the sensitive film when the gas is measured is dependent on the surface state (initial state) of the sensitive film before measuring the gas. That is, the recent history of the sensitive film before measuring a certain gas will affect the detection, and the detection accuracy may deteriorate if this is not taken into account.

With regard to this problem, according to JP-A No. 9-264591, the detection accuracy is improved by measuring a change in a physical value at different temperatures and calculating a difference (hysteresis) of the change.

However, it is predicted that when the concentration of the gas is low, the difference is not clearly shown and the detection accuracy is lowered when the concentration of the gas is low. The response speed is lowered since the temperature must be elevated and lowered repeatedly to calculate the difference.

Among gas sensors, there is most widely used a gas sensor utilizing a metal oxide semiconductor such as SnO₂, ZnO, In₂O₃ or the like as a sensitive film.

Such sensors can be classified into, for example, a sintered body type, a thick film type, a thin film type and the like by a method of fabricating the sensitive film and a thickness thereof. Among them, according to a thin film type gas sensor a sensitive film of which comprises a thin film, since the sensitive film comprises the thin film, a gas adsorbed to the surface of the sensitive film can be diffused to a total of the sensitive film in a short period of time. Therefore, it is expected that the response speed is larger and the sensitivity is higher than those of the sintered body type or the thick film type gas sensors.

In such a thin film type sensor, the sensitive film, which is a thin film, is formed on, for example, an insulating substrate by a vacuum deposition process, a sputtering process or an ion plating process and a pair of electrodes are formed above the sensitive film. The change in a physical value of the sensitive film, when the sensitive film is exposed to a gas to be detected, is detected from the electrodes as an electric signal, and the kind of gas or the concentration of the gas are specified from the change in the physical value.

However, according to the above-mentioned method of forming the sensitive film, the metal oxide semiconductor is liable to inlclude fine crystals. As a result, in the sensitive film, the gas to be detected is diffused through a very small crystal boundary and therefore, actually, the time period necessary for diffusing or removing the gas to be detected becomes as long as about several minutes and the response is worse in comparison with the gas sensor of the sintered body type.

The change in the physical value of the sensitive film is dependent on the temperature of the sensitive film and the dependency of the change in the physical value on the temperature differs by the kind of the gas being detected. Therefore, normally, the temperature of the sensitive film is set to various temperatures between about 300 to 450° C. and the kind of the gas and the concentration of the gas are specified by measuring the change in the physical value at that occasion. However, according to the thin film type gas sensor comprising the small crystals, grain growth is progressed by the heating operation and stability of the sensitive film is poor with age and the detection accuracy deteriorates.

With regard to this problem, according to JP-A No. 8-94560, a sensitive film of a single crystal is formed by epitaxially growing a single crystal of the sensitive film on an insulating substrate by reactive sputtering. As a result, the crystal grains are enlarged and crystal grains are reduced, which improves response.

However, according to this patent publication, the sensitive film is epitaxially made to grow to succeed the single crystal structure of the substrate by using a single crystal insulating substrate (sapphire or the like) to reduce grain boundaries in the sensitive film, and therefore, the boundaries of the sensitive film cannot be reduced unless a limited material of the single crystal insulating substrate is used.

### SUMMARY OF THE INVENTION

Basically, the invention provides a thin-film type gas sensor capable of improving response regardless of the kind of substrate used and a method of fabricating the same. The invention provides a gas sensor capable of improving reliability and a method of fabricating the same. It is an object of the invention to provide a method of fabricating a gas sensor having a reduced number of fabricating steps. Further, it is an object of the invention to provide a method of detecting a gas by using a gas sensor capable of identifying the gas with high accuracy.

According to an aspect of the invention, there is provided a gas sensor that includes a substrate (1), a heater layer (3) formed above the substrate, an electrically insulating heater insulation layer (4) formed above the heater layer and the substrate, a detection electrode (6a, 6b) formed above the heater insulator layer, a flattened electrically insulating layer (9) formed above the heater insulator layer and around the detection electrode such that a surface of the detection electrode is exposed. A surface of the heater insulation layer is flattened to be flush with the surface of the detection electrode. A sensitive film (5) is formed flatly in contact with the surface of the detection electrode. A physical value of the film is changed by reacting with the gas to be detected.

According to the aspect of the invention, the detection electrode for detecting a change in the physical value of the sensitive film is located below the sensitive film and the flattened insulating layer fills the surroundings of the detection electrode. Therefore, recesses or projections, or steps, caused by the detection electrode are reduced or eliminated, and the sensitive film can be formed on the flattened face. Therefore, the gas sensor inhibits the sensitive film from being broken and is reliable.

According to another aspect of the invention, a gas sensor includes a substrate (1), a heater layer (3) formed above the substrate, a detection electrode (6a, 6b) formed on a face, which is the same that the heater layer is on. The detection electrode is electrically insulated from the heater layer. A flattened electrically insulating layer (9) is formed above the heater layer to cover the heater layer. A surface of the insulating layer (9) is flattened to be flush with a surface of the detection electrode such that the surface of the detection electrode is exposed. A sensitive film (5) is formed flatly in contact with the surface of the detection electrode above the flattened electrically insulating layer. A physical value of the film is changed by reacting with the gas to be detected.

The sensitive film is formed above the flattened electrically insulating layer and therefore, the gas sensor inhibits the sensitive film from being broken, which improves reliability.

According to another aspect of the invention, the gas sensor includes a substrate (1), an electrically insulating layer (31) formed above the substrate, and a sensitive film (5) formed flatly above the electrically insulating layer. A physical value of the film is changed by reacting with the gas to be detected. A heater layer (3) is located between the substrate and the electrically insulating layer to surround the sensitive film and not directly below the sensitive film. A detection electrode (6a, 6b) is formed above the sensitive film for detecting a change in a physical value of the sensitive film.

The detection electrode is formed above the sensitive film, and the heater layer is not directly below the sensitive film. Therefore, the sensitive film can be formed on a flat face that is free of recesses and projections, or steps caused by differences in elevations. Therefore, the gas sensor prevents or limits breakage of the sensitive film.

When the surface of the electric insulating layer that contacts the sensitive film is flattened such that the maximum step elevation, or difference between a high point and a low point of the surface, is smaller than the film thickness of the sensitive film, breakage of the sensitive film can be inhibited.

According to another aspect of the invention, the heater layer has the shape of a frame, and a temperature control film (41) for facilitating heat transfer from the heater layer is formed as a flattened film on the face that the heater layer is on. The temperature control film is on an inner side of the heater layer, and an outer periphery of the temperature control film is arranged between the inner periphery of the heater layer and the outer periphery of the sensitive film when viewed from above the sensitive film.

By providing the temperature control film in this way, the temperature uniformity of the sensitive film can be improved. Since the temperature control film is larger than the sensitive film, the sensitive film can be formed on a flat face and the sensitive film can be prevented from being broken.

The corners of the heater layer can be chamfered or rounded.

Generally, it is preferred that lines of equal temperature (isotherms) do not have angles and are formed in rounded shapes. Therefore, when the corner portion of the heater layer is chamfered or rounded, the shape of the heater layer can be matched to shapes of the isotherms, and temperature control becomes easier.

The sensitive film can be oval.

Generally, the temperature distribution in the sensitive film depends on the distance from the heater layer. Therefore, by forming the sensitive film in the shape of an oval or a circle, parts of the sensitive film remote from the heater layer can be eliminated, and deviation in the temperature distribution of the sensitive film can be reduced.

As described above, it is normal that the isotherms are rounded, and therefore, when the corners of the sensitive film are chamfered or rounded, the shape of the sensitive film matches the shapes of the isotherms and the temperature control becomes easier.

According to another aspect of the invention, a support film (2) is formed above the substrate and the heater layer is formed above the support film, a hollow cavity (8) is formed in the substrate below the heater layer and the sensitive film and the hollow portion are bridged by the support film. The tensile stress applied to the support film is equal to or larger than 40 MPa and equal to or smaller than 150 MPa.

According to the aspect of the invention, by forming the hollow cavity below the heater layer and the sensitive film, heat transfer to the substrate is impeded and the temperature of the sensitive film can be more easily increased, and power consumption is reduced. In the case of forming the hollow cavity, when compressive stress is applied to the support film, the support film is damaged, however, since light tensile stress is applied to the support film, the support film can be inhibited from being broken.

The heater layer is arranged between an outer periphery of the hollow cavity and the outer periphery of the sensitive film.

Thus, the heater layer is not located directly below the sensitive film, and the sensitive film can be warmed from its periphery. When the heater layer is formed above the hollow portion, heat transfer from the heater layer is limited.

The outer periphery of the hollow cavity at a surface of the substrate and the outer periphery of the sensitive film are formed by shapes similar to each other when viewed from above the sensitive film.

Generally, the isotherms in the sensitive film are dependent on the shapes of the hollow cavity, the heater layer and the sensitive film. By forming the outer peripheries of the hollow cavity, the heater layer and the sensitive film with similar shapes, the isotherms in the support film and the sensitive film above the hollow cavity will be concentric, and temperature control of the sensitive film will be easier.

When the surface of the detection electrode exposed from the flattened insulating layer and the surface of the flattened electrically insulating layer have different elevations such that a step is formed, and when the maximum step elevation is smaller than the film thickness of the sensitive film, breakage of the sensitive film can be inhibited.

According to another aspect of the invention, a support film (2) is formed above the substrate and the heater layer is formed above the support film, and a hollow cavity (8) is formed in the substrate below the heater layer and the sensitive film, the hollow cavity is bridged by the support film, and tensile stress equal to or larger than 40 MPa and equal to or smaller than 150 MPa is applied to the sensitive film.

Thus, the temperature of the sensitive film can easily be raised and power consumption is reduced. In the case of forming the hollow cavity, when compressive stress is applied to the support film, the support film may break, however, since light tensile stress is applied to the support film, the support film is inhibited from being broken.

The total of stresses on the support film and all members formed above the support film is equal to or larger than 40 MPa and equal to or smaller than 150 MPa.

Generally, when compressive stress is applied to the support film formed above the hollow cavity, the support film will break, however, by imposing tensile stress on the film and the heater layer and the like above the hollow cavity, breakage of the support film is inhibited.

A projected portion (51) is formed at the support film on a side of the hollow cavity. By providing the projected portion by, for example, leaving a portion of the substrate at a location of the support film at which the temperature is liable to rise, heat transfer can be improved and temperature control of the sensitive film is easier.

According to another aspect of the invention, the gas sensor is for detecting gas at room temperature, and the gas sensor includes an electrically insulating substrate (1) and a sensitive film (5) formed above the substrate. A physical value of the film changes by reacting with the gas to be detected. A detection electrode (6a, 6b) is formed above the sensitive film for detecting a change in the physical value of the sensitive film.

In the case of the gas sensor for detecting the gas at room temperature, since the heater layer is not necessary, by using the electrically insulating substrate and using the sensitive film sensitive to the gas to be detected at the room temperature, the sensitive film can be formed at a face above the substrate that has no steps (recesses or projections). Therefore, the gas sensor inhibits the sensitive film from being broken, which improves reliability.

When a filter (12) for permeating only a specific gas is provided above the sensitive film, the selectivity of the specific gas is improved.

The thickness of the sensitive film is equal to or larger than 3 nm and equal to or smaller than 12 nm.

By sizing the sensitive film in this way, response speed can be improved by restraining in-film diffusion of the gas to be detected in the sensitive film.

A fabrication method is summarized as follows. The gas sensor can be fabricated by forming a heater layer (3) above a substrate (1); forming a first electrically insulating layer (4) above the heater layer and the substrate; forming a detection electrode (6a, 6b) above the first electrically insulating layer; forming a second electrically insulating layer (9a) above the first electrically insulating layer to cover the detection electrode; flattening and thinning the second electrically insulating layer until a surface of the detection electrode is exposed; forming a sensitive film (5), a physical value of which is changed by reacting with a gas to be detected, above the flattened second electrically insulating layer to cover the exposed detection electrode; and electrically connecting the detection electrode and the sensitive film.

A heater layer (3) and a detection electrode (6a, 6b) are simultaneously formed on the same face above a substrate (1) with different thicknesses. An electrical insulating layer (9b) is formed above the substrate to cover the heater layer and the detection electrode. The method includes flattening and thinning the electrically insulating layer until a surface of the detection electrode is exposed. And a sensitive film (5), which reacts to a gas to be detected, is formed above the electrically insulating the layer to cover the exposed detection electrode. The detection electrode is electrically connected to the sensitive film.

Since the heater layer and the detection electrode are formed simultaneously on the same face, this method of fabricating the gas sensor reduces the number of fabricating steps.

In the heater layer and the detection electrode forming step, a metal thin film (21) for finalizing the heater layer and the detection electrode is formed above the substrate, and a photoresist (22) is formed above the metal thin film. At a portion of the photoresist in correspondence with the heater layer, by developing the photoresist with a photo mask (23) having a fine pattern (23b) equal to or smaller than the resolution of the exposure apparatus being employed, a pattern in which the thickness of a portion (22b) in correspondence with the heater layer is thinner than a portion (22a) corresponding to the detection electrode is formed in the photoresist. By etching the metal thin film with the photoresist, which is formed with the different thicknesses, the thickness of the heater layer is made smaller than the thickness of the detection electrode.

The method includes forming a heater layer (3) above a substrate (1); forming an electrically insulating layer (31) above the heater layer; forming a sensitive film (5), which reacts to a gas being detected, above the electrically insulating layer and not directly above the heater layer; and forming a detection electrode (6a, 6b), which detects changes in the sensitive film, above the sensitive film.

The method can include forming a support film (2) between the substrate and the heater layer; forming a mask (11), which has an opening portion (11a) at a location of the substrate in correspondence with a lower side of the sensitive film, on a face of the substrate opposite to the sensitive film, and forming a hollow cavity (8) in correspondence with the opening portion by etching the substrate via the mask.

In the mask forming step, a central portion (11b) can be covered, and, when the hollow cavity is formed, a projection is left after etching the substrate via the mask.

The method can include forming heater pads (7c, 7d) and detection electrode pads (7a, 7b). Further, the method can include forming a filter (12) for permitting passage of only a specific gas to the sensitive film after the pad forming step. This method includes removing the filter above the pads after the hollow cavity is formed.

Another aspect of the invention is a method of detecting a gas by using a gas sensor that includes a substrate (101) and a sensitive film (105) formed on the substrate. A physical value of the film changes in response to absorbing and desorbing the gas. A heater (103) is formed on the substrate for controlling the temperature of the sensitive film. The sensor includes detecting means (106a, 106b) for detecting a change in the physical value of the sensitive film, heater control means (201) for controlling the temperature of the heater, and analyzing means (202) for analyzing the change in the physical value of the sensitive film. At least one of the identity and the concentration of the gas is determined by changing the temperature of the heater to a plurality of temperatures (H1 through H6) to set the temperature of the sensitive film to a plurality of detection temperatures. The temperature of the sensitive film is set temporarily to a predetermined temperature before detecting the change in the physical value of the sensitive film.

Accordingly, the sensitive film returns to a predetermined state temporarily. Thus, the influence of the history of the sensitive film on the detection when the temperature of the sensitive film is changed to a plurality of the detection temperatures does not affect the change in the physical value of the sensitive film. Therefore, the gas sensor is highly accurate.

According to another aspect of the invention, a method of detecting a gas employs a gas sensor that includes a substrate (101), a sensitive film (105), a heater (103), detecting means (106a, 106b), heater controlling means (201) and analyzing means (202). At least one of the identity of a component gas and the concentration of the gas being detected is determined by repeatedly changing the temperature of the heater to a constant temperature (H7) to set the temperature of the sensitive film repeatedly to a constant detection temperature and by detecting the change in the physical value of the sensitive film at the constant detection temperature. The temperature of the sensitive film is temporarily set to a predetermined temperature before detecting the change in the physical value of the sensitive film.

Accordingly, the influence of the history of the sensitive film does not affect the change in the physical value of the sensitive film. Therefore, accuracy is improved.

When the temperature of the sensitive film is set to the plurality of detection temperatures, the temperature of the sensitive film is temporarily set to the predetermined temperature every time before the temperature of the sensitive film is set to the respective detection temperatures.

Thus, every time the change in the physical value is detected, the change in the physical value can always be detected as a change from the predetermined reference value, and the gas can be detected with higher accuracy.

By making the predetermined temperature higher than the detection temperature or detection temperatures, desorption of gases or moisture present on the surface of the sensitive film is improved and the surface state of the sensitive film can be brought into a predetermined initial state in a short period of time. Thus, the sensor is fast and accurate. By setting the predetermined temperature to be equal to or higher than the temperature at which a gas that has been adsorbed in the sensitive film is desorbed from the sensitive film, at least the gas adsorbed to the sensitive film can be desorbed. Therefore, a state in which the gas is not adsorbed by the sensitive film is the initial state.

By setting the predetermined temperature to be equal to or higher than the temperature at which moisture adsorbed to the sensitive film is desorbed from the sensitive film, at least moisture adsorbed to the surface of the sensitive film can be desorbed. Therefore, a state in which moisture is not adsorbed to the sensitive film is the initial state.

By setting the predetermined temperature to be equal to or higher than a temperature at which the sensitive film does not cause a change in the physical value by adsorbing the gas, the initial state is a state of the sensitive film in which the physical value does not change.

By maintaining the sensitive film at the predetermined temperature for a predetermined period of time, gases or moisture is positively desorbed from the sensitive film.

When gas or moisture is completely desorbed from the sensitive film, the change in the physical value of the sensitive film is stable, and the temperature of the sensitive film can be set to the detection temperature after confirming that gas or moisture is desorbed from the sensitive film by setting the sensitive film to the predetermined temperature and setting the temperature of the sensitive film to the detection temperature after the change in the physical value of the sensitive film has stabilized.

The change in the physical value of the sensitive film is detected after setting the temperature of the sensitive film to the detection temperature and maintaining the temperature at the detection temperature for a predetermined period of time.

Thus, the change in the physical value of the sensitive film can be detected after adsorption of the gas to the sensitive film has progressed, and therefore, the gas can be detected with high accuracy.

When the gas is sufficiently adsorbed in the sensitive film, the change in the physical value of the sensitive film is stabilized, and therefore, the temperature of the sensitive film is set to the detection temperature after confirming that the gas has been adsorbed to the sensitive film, by detecting the change in the physical value of the sensitive film after the temperature of the detection film is set to the detection temperature and after the change in the physical value of the sensitive film has stabilized.

The change in the physical value of the sensitive film can be detected before the change in the physical value is stabilized.

Generally, the slope of the change in the physical value of the sensitive film differs in accordance with, for example, the concentration of the gas, and therefore, the gas can be identified even before the change in the physical value of the sensitive film has stabilized. Therefore, the gas to be detected can be identified in a short period of time, and therefore, the gas can be identified with high accuracy and high response speed.

The temperature of the heater is made lower than the lowest ignition temperature conceivable in the environment of the gas sensor.

Thus, it is not necessary to provide a combustion-proof construction for the gas sensor.

By forming a hollow cavity (108) at the substrate, a thin-walled portion can be formed at a portion of the substrate in correspondence with the hollow cavity, and the heater and the sensitive film are formed at the thin-walled portion. Such a thin-walled portion has a small thermal capacity and high insulating performance, and therefore, power consumption is reduced, and the temperature of the sensitive film can be changed with high response.

The sensitive film may be a thin film having a thickness equal to or smaller than 10 nm. By sizing the sensitive film in this way, diffusion of the gas to an inner portion of the sensitive film can be prevented, and the response of the gas sensor improves.

Electric resistance can be detected as the change in the physical value.

According to another aspect of the invention, a thin-film type gas sensor having a substrate (301) and a sensitive film (302) formed above the substrate. The sensitive film has an average crystal grain diameter equal to or larger than the film thickness of the sensitive film.

According to the aspect of the invention, when various substrates are used, the crystal grain boundary in the sensitive film can be reduced by making the average crystal grain diameter of the sensitive film equal to or larger than the film thickness of the sensitive film by controlling the composition of the sensitive film. This improves response regardless of the type of substrate that is used.

Specifically, an alumina substrate or a mullite substrate can be used. When the height of any projection from the surface of the substrate and the depth of any recess from the surface of the substrate is equal to or less than ⅕ of the film thickness of the sensitive film, the average crystal grain diameter of the sensitive film can preferably be made equal to or larger than the film thickness of the sensitive film.

When using a silicon substrate, when the sensitive film is formed above the substrate via an insulating substance (305), effective electric insulation is ensured between the silicon substrate and the sensitive film.

When the insulating substance on the silicon substrate is formed as a single crystal, the crystal grain diameter of the sensitive film can further be enlarged by succeeding the single crystal structure of the insulating substance.

It is preferred that the insulating substance includes at least one of CaF₂, Al₂O₃ and CeO₂.

When the film thickness of the sensitive film is equal to or smaller than the thickness of a depletion layer produced by adsorbing the gas to be detected to the sensitive film, detection sensitivity and response can further be improved. It is preferred that the film thickness of the sensitive film is equal to or larger than 3 nm and equal to or smaller than 12 nm.

The invention may include a heater layer (304) for heating the sensitive film formed above the substrate, and a portion of the substrate in correspondence with and below the sensitive film is constructed by a thin-walled structure, the thickness of which is smaller than that of the remainder of the substrate.

Thus, heat transfer from the heater layer via the substrate can be reduced. Therefore, the thin-film type gas sensor reduces power consumption while maintaining high response.

By forming a filter layer (311), which selectively permits a gas to be detected, above the sensitive film, the selectivity of the sensor is improved.

It is preferred that the film thickness of the heater layer in this case be equal to or larger than 10 nm or equal to or smaller than 50 nm.

The invention includes a method of fabricating a thin-film type gas sensor. The method includes forming a sensitive film (302), which reacts to a gas to be detected, above a substrate (301). The method includes reducing recesses and projections in the surface of the substrate to dimensions equal to or less than ⅕ of the film thickness of the sensitive film and forming a sensitive film that has an average crystal grain diameter equal to or larger than the film thickness by depositing the sensitive film above the substrate by an atomic layer growing method.

By depositing the sensitive film with an atomic layer growing method, even when a substrate that is not provided with a single crystal structure is used, the sensitive film can be formed nearly stoichiometrically. As a result, the crystal grain diameter of the sensitive film will be large. Since the crystal grain boundary is thus reduced, the method of fabricating the thin-film type gas sensor will improve the sensor response regardless of the type of substrate that is employed.

The invention includes a method of fabricating a thin-film type gas sensor having a sensitive film (302), a physical value of which is changed by reacting with a gas to be detected, on a substrate (1). The method includes forming the sensitive film above the substrate, and forming an insulating layer (307) at a middle section of the sensitive film, such that the insulating layer is substantially parallel with the substrate, by implanting ions in the sensitive film. In the ion implanting step, the position of the insulating layer in the sensitive film is adjusted such that in a sensitive film upper layer portion (302a) of the sensitive film located above the insulating layer, and the average crystal grain diameter of the sensitive film upper layer portion becomes equal to or larger than the film thickness of the sensitive film upper layer portion.

Thus, even when the average crystal grain diameter of the sensitive film is small, due to the insulating layer formed by the ion implanting step, the film thickness of the sensitive upper layer portion can be made equal to or smaller than the average grain diameter. Since the sensitive film upper layer portion functions as the sensitive film, the average crystal grain diameter can be made equal to or larger than the film thickness, and therefore, the crystal grain boundary at the sensitive film upper layer 2a can be reduced. As a result, the thin-film type gas sensor is more responsive regardless of the substrate that is used.

The invention includes a method of fabricating a thin-film type gas sensor having a sensitive film (302), a physical value of which is changed by reacting with a gas to be detected, above a substrate (301). The method includes forming the sensitive film above the substrate, forming an ion-implanted layer (307) at a middle section in the sensitive film parallel with the substrate by implanting ions in the sensitive film, and dividing the sensitive film at the ion-implanted layer by heat treating the ion-implanted layer. In the ion implanting step, the position of the ion-implanted layer in the sensitive film is adjusted such that an average crystal grain diameter becomes equal to or larger than the film thickness at least one of a sensitive film upper layer portion (302a) of the sensitive film above the ion-implanted layer and a sensitive film lower layer portion (302b) in the sensitive film, which is located below the ion-implanted layer.

At least one of the sensitive film upper layer portion and the sensitive film lower layer portion, after division, can be used as a layer for absorbing and desorbing the gas to be detected. The layer is formed such that the average grain diameter is larger than the film thickness, and therefore, responsiveness is improved regardless of the kind of substrate used.

When, in the sensitive film forming step, the sensitive film is formed by alternately supplying a gas that includes a metal and water, the average crystal grain diameter can be made larger than the film thickness of the sensitive film.

The sensitive film is formed by an atomic layer growing method.

In the atomic layer growing method, the composition of the metal oxide can be controlled with extremely high accuracy, and therefore, the average crystal grain diameter of the sensitive film can effectively be made larger than the film thickness of the sensitive film.

The sensitive film may be formed above the substrate via an insulating substance (305), and the insulating substance is formed by the atomic layer growing method.

For example, when the insulating performance of the substrate is insufficient, the insulating film may be formed above the substrate. By forming the insulating substance by the atomic layer growing method, the composition of the sensitive film can be controlled with extremely high accuracy.

Further, the invention may include forming a filter layer (311), which selectively permits the gas being detected to reach the sensitive film, by the atomic layer growing method after the sensitive film is formed.

By forming the filter layer by the atomic layer growing method, the surface of the sensitive film can be firmly covered with the thin film of the filter. Therefore, it is not necessary to thicken the filter layer to firmly cover the surface of the sensitive film and the thin-film type gas sensor will be highly responsive and selective.

### BRIEF DESCRIPTION OF THE SEVERAL VIEWS OF THE DRAWING

FIG. 1 is a plan view of a gas sensor according to a first embodiment of the invention;

FIG. 2 is a cross sectional view taken long a line 2-2 of FIG. 1;

FIGS. 3A, 3B and 3C are diagrammatic cross sectional views showing consecutive stages of a method of fabricating the gas sensor according to the first embodiment;

FIGS. 4A, 4B and 4C are diagrammatic cross sectional views showing steps that occur after the stage illustrated in FIG. 3C;

FIG. 5 is a diagrammatic cross sectional view of a gas sensor according to a second embodiment taken along line 2-2 of FIG. 1;

FIGS. 6A, 6B and 6C are diagrammatic cross sectional views showing stages of a method of fabricating a gas sensor according to the second embodiment;

FIGS. 7A, 7B and 7C are diagrammatic cross sectional views showing stages of the method of the second embodiment subsequent to the stage illustrated in FIG. 6C;

FIGS. 8A, 8B, 8C and 8D are diagrammatic cross sectional views showing in detail steps of forming a heater layer in the process of fabricating the gas sensor according to the second embodiment;

FIG. 9 is a diagrammatic plan view of a photoresist used in the method of the second embodiment;

FIG. 10A is an enlarged plan view of a part of FIG. 9 demarcated by a box C in FIG. 9;

FIG. 10B is a graph indicating the level of transmitted light in relation to corresponding positions of FIG. 10A;

FIG. 10C is a diagrammatic cross sectional view of the photoresist layer 22 that corresponds to the box C after development that shows a relationship to FIGS. 10A and 10B for illustrating a method of varying the film thickness of the photoresist in the method of the second embodiment;

FIG. 11 is a diagrammatic plan view of a gas sensor according to a third embodiment;

FIG. 12 is a diagrammatic cross sectional view taken along a line 12-12 of FIG. 11;

FIG. 13A, 13B and 13C are diagrammatic cross sectional views showing stages of a method of fabricating the gas sensor according to the third embodiment;

FIGS. 14A, 14B and 14C are diagrammatic cross sectional views showing stage of the method of the third embodiment subsequent to the stage illustrated in FIG. 13C;

FIG. 15 is a plan view of a gas sensor according to a fourth embodiment;

FIG. 16 is a plan view of a gas sensor according to a fifth embodiment;

FIG. 17 is a plan view of a gas sensor according to a sixth embodiment;

FIG. 18 is diagrammatic cross sectional view of a section taken along a line 18-18 of FIG. 17;

FIG. 19 is a plan view of a gas sensor according to a seventh embodiment;

FIG. 20 a plan view of other gas sensor according to the seventh embodiment;

FIG. 21 is a plan view of a gas sensor according to an eighth embodiment;

FIG. 22 is a plan view of other gas sensor according to the eighth embodiment;

FIG. 23 is a plan view of a gas sensor according to a ninth embodiment;

FIG. 24 is a diagrammatic cross sectional view of a gas sensor according to a tenth embodiment;

FIG. 25 is a plan view of a gas sensor according to an eleventh embodiment;

FIG. 26 is a diagrammatic cross sectional view taken long a line 26-26 of FIG. 25;

FIG. 27 is a diagrammatic cross sectional view illustrating a method of fabricating the gas sensor according to the eleventh embodiment;

FIG. 28 is a diagrammatic cross sectional view of a gas sensor according to a twelfth embodiment;

FIG. 29 is a diagrammatic cross sectional view of a gas sensor according to a thirteenth embodiment;

FIG. 30 is a diagrammatic plan view showing a gas sensor according to a fourteenth embodiment of the invention;

FIG. 31 is a diagrammatic cross sectional view of a section taken along a line 31-31 of FIG. 30;

FIG. 32 is a three-dimensional graph showing dependencies of the sensitivity of a sensitive film for various gases on temperature of the sensitive film;

FIG. 33 is a pair of graphs showing a relationship between heater temperature and resistance of the sensitive film for various gasses according to the fourteenth embodiment of the invention;

FIG. 34 is a pair of graphs showing an enlargement of a part of the graphs in FIG. 33;

FIG. 35 is a pair of graphs showing a relationship between the temperature of a heater and the change in resistance of a sensitive film according to a fifteenth embodiment of the invention;

FIG. 36 is a perspective view of a bridge-type gas sensor;

FIG. 37 is a perspective view of a gas sensor according to a seventeenth embodiment;

FIG. 38 is a diagrammatic cross sectional view taken along a line 38-38 of FIG. 37;

FIG. 39 is a view like FIG. 38 showing an enlargement of a part of FIG. 38;

FIG. 40 is an enlarged diagrammatic cross sectional view of a comparative example in which a sensitive film is formed by small crystals;

FIG. 41 is a graph showing a change of resistivity when the film thickness of the sensitive film is changed over time according to the seventeenth embodiment;

FIG. 42 is diagrammatic cross sectional view of a gas sensor according to an eighteenth embodiment;

FIG. 43 is a diagrammatic cross sectional view of a gas sensor according to a nineteenth embodiment;

FIGS. 44A, 44B, 44C and 44D are diagrammatic cross sectional views showing stages of a method of fabricating the gas sensor according to the nineteenth embodiment;

FIGS. 45A, 45B, 45C and 45D are diagrammatic cross sectional views showing a method of fabricating a gas sensor according to a twenty-first embodiment;

FIGS. 46A, 46B and 46C are diagrammatic cross sectional views showing stages of a method of fabricating a gas sensor according to a twenty-second embodiment;

FIGS. 47A and 47B are diagrammatic cross sectional views showing stages that are subsequent to that of FIG. 46C;

FIG. 48 is a diagrammatic cross sectional view of a gas sensor according to a twenty-third embodiment; and

FIG. 49 is a diagrammatic cross sectional view of a conventional gas sensor.

### DETAILED DESCRIPTION OF THE PREFERRED EMBODIMENTS

### First Embodiment

As shown by FIG. 2, a support film 2 is formed on a substrate 1. The substrate 1 is a semiconductor substrate including, for example, silicon (Si) or the like. The support film 2 is formed by laminating together a silicon oxide film and a silicon nitride film.

A heater electrode 3 forms a heater layer on the support film 2. The heater electrode 3 is for warming a sensitive film 5, which is discussed later, to, for example, about 500° C. The width of the heater electrode 3 is as small as possible, and the length of the heater electrode 3 is as long as possible for facilitating heat generation. The heater electrode 3 is arranged at an area corresponding with and right below the sensitive film 5 for uniformly heating the sensitive film 5.

Specifically, the heater electrode 3 meanders directly below the sensitive film 5. Both ends of the heater electrode 3 extend to peripheral portions of the substrate 1. The heater electrode 3 can be made by a noble metal substance of platinum (Pt), gold (Au), RuO₂, polysilicon or the like.

A lower electrically insulating layer 4 for insulating the heater is formed above the heater electrode 3 and the support film 2. The insulating layer 4 is a combination of a silicon oxide film and a silicon nitride film. Ideally, the support film 2 and the lower insulating layer 4 are symmetrical with respect to the heater electrode 3. For example, when the support film 2 is made by laminating the silicon oxide film on the silicon nitride film, the lower insulating layer 4 is made by laminating the silicon nitride film on the silicon oxide film.

The support film 2 and the lower insulating layer 4 span a hollow cavity 8 formed in the substrate. By warming the support film 2 and the lower insulating layer 4 with the heater electrode 3, the support film 2 and the lower insulating layer 4 might be deformed by a difference in degree of thermal expansion of the silicon oxide film and the silicon nitride film. However, when the support film 2 and the lower insulating layer 4 are symmetrically formed, the deformation is countered.

An upper face of the lower insulating layer 4 is made flat. In order to make the upper face flat, the lower insulating layer 4 may be polished by CMP (Chemical Mechanical Polishing) or the like, or when the lower insulating layer 4 is formed, conditions of pressure, temperature, composition ratio of gas and the like may be set such that the upper face is flatly formed. The upper face may be made flat by using a spin-on-glass process or the like.

Two detection electrodes 6a, 6b detect changes in a physical value of the sensitive film 5. In this embodiment, the physical value of the sensitive film 5 is resistance. Each of the detection electrodes 6a, 6b is formed in a comb-like shape. When viewed from above the gas sensor, the comb teeth portions of the detection electrodes 6a, 6b are arranged at turn intervals of the heater electrodes 3, which meander. The ends of the respective detection electrodes 6a, 6b extend toward the periphery of the substrate 1. Detection electrode pads 7a and 7b are formed at the ends of the respective detection electrodes 6a, 6b.

The detection electrodes 6a, 6b are made of material including a noble metal (such as platinum (Pt) or gold (Au)), tungsten (W), titanium (Ti), aluminum (Al) or the like. An alloy of these may also be used. The pads 7a and 7b may be made of, for example, aluminum, gold or the like. As mentioned later, a material having strength for adhering to bonding wires is formed at the pads 7a and 7b.

A flattened upper electrically insulating layer 9 is formed above the lower insulating layer 4 in space surrounding the detection electrodes 6a, 6b, such that upper surfaces of the detection electrodes 6a, 6b are exposed. The surface of the upper insulating layer 9 and that of the detection electrodes 6a, 6b are flattened and made flush.

That is, the upper insulating layer 9 fills space surrounding the detection electrodes 6a, 6b such that the surfaces of the detection electrodes 6a, 6b and the surface of the upper insulating layer 9 are flush. A film that is a combination of, for example, a silicon oxide film, a silicon nitride film or the like can be used for the upper insulating layer 9.

The sensitive film 5 is formed flatly in contact with the surfaces of the detection electrodes 6a, 6b above the upper insulating layer 9. The sensitive film 5 reacts with a gas to be detected and the resistance of the sensitive film 5 changes accordingly. The sensitive film 5 may be made of an oxide semiconductor material such as SnO₂, TiO₂, ZnO and In₂O₃. The sensitive film 5 may be formed with a thickness of about several nanometers. Specifically, it is preferred that the thickness of the sensitive film 5 be equal to or larger than 3 nm and equal to or smaller than 12 nm.

By setting the thickness of the sensitive film 5 in this way, the response speed can be improved by reducing the time period during which the gas to be detected can diffuse into the sensitive film 5 by inhibiting the gas to be detected from diffusing to an inner portion of the sensitive film 5. When the thickness of the sensitive film 5 is set to the same thickness as the depletion layer produced by adsorbing the gas to be detected in the sensitive film 5, a large sensitivity can be provided while providing high responsiveness. Depending on the kind of the gas, the sensitivity of the sensor to the gas may be improved by adding an impurity to the sensitive film 5.

On upper sides of both ends of the heater electrode 3 at the peripheral portions of the substrate 1, openings are formed in the lower insulating layer 4 and the upper insulating layer 9 to make electrode lead-out ports 4a. Heater pads 7c and 7d are formed on the surface of the upper insulating layer 9 at the electrode lead-ports 4a and are electrically connected to the heater electrode 3. The heater pads 7c and 7d can be made of a material that is the same as that of the detection electrode pads 7a and 7b.

The hollow cavity 8 is formed below the heater electrode 3, the detection electrodes 6a, 6b and the sensitive film 5 in the substrate 1. The hollow cavity 8 is opened in a lower direction of the substrate 1, as viewed in FIG. 2, and is bridged by the support film 2 on the upper face of the substrate 1.

When compressive stress is applied to the support film 2, the support film 2 may be damaged. Therefore, the support film 2 is provided with light tensile stress overall. In detail, the silicon oxide film is provided with compressive stress, the silicon nitride film is provided with tensile stress, and the support film 2 is provided with net light tensile stress by adjusting the film thicknesses.

With regard to specific tensile stress, it is known that when, a support film 2 under tensile stress of 30 MPa is heated to about 200° C., the support film is damaged. Therefore, it is preferred to impose a tensile stress equal to or larger than 40 MPa and equal to or smaller than 150 MPa.

Damage to the support film 2 can be prevented with more certainty when, the total of the stresses of the support film 2 and all the members formed above the support film 2 (heater electrode 3, lower insulating layer 4, detection electrodes 6a, 6b, upper insulating layer 9 and sensitive film 5) is a tensile stress equal to or larger than 40 MPa and equal to or small than 150 MPa. Although not illustrated, by electrically connecting, for example, bonding wires to the detection electrode pads 7a and 7b and the heater pads 7c and 7d, circuits can be completed for electric transmission and reception of information from the detection electrodes 6a, 6b and activation of the heater electrode 3.

The sensitive film 5 is set to various temperatures of about 300° C. through 500° C. by generating heat with the heater electrode 3, and changes in the resistance of the sensitive film 5 at the respective temperatures, are detected by the detection electrodes 6a, 6b. The changes in the resistance of the sensitive film 5 at the respective temperatures depend on the kind and the concentration of the gas to be detected. Further, the temperature dependency of the change in the resistance of the sensitive film 5 differs by the kind of the gas to be detected. Therefore, the kind and the concentration of the gas to be detected can be detected by detecting the changes in the resistance of the sensitive film 5 at various temperatures.

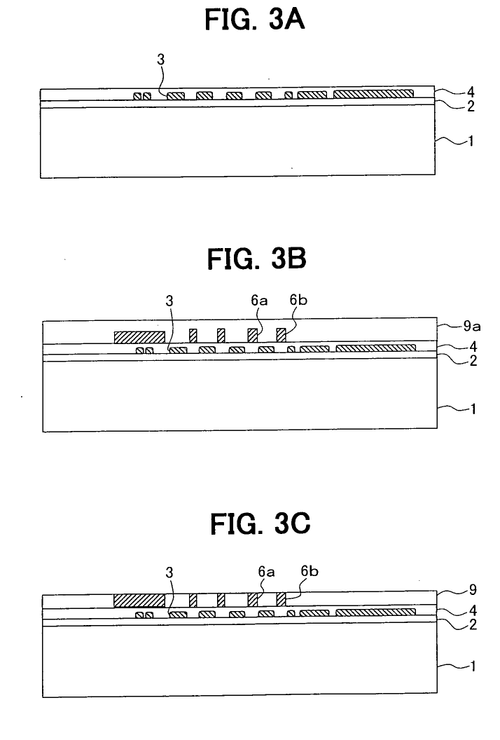

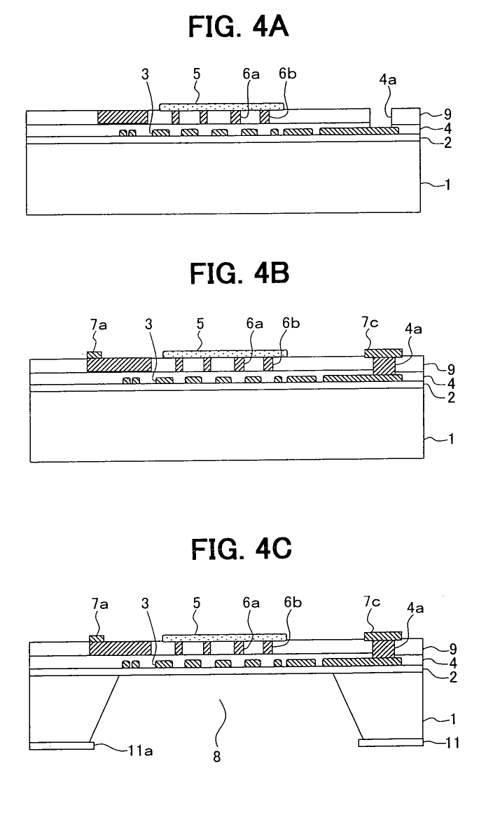

Next, a method of fabricating the gas sensor will be described with reference to FIGS. 3A, 3B, 3C, 4A, 4B and 4C

Step of FIG. 3A

First, the substrate 1 is prepared and the support film 2 is formed on the substrate 1 by a thermal oxidation process, a plasma CVD process or an LP-CVD process. The heater electrode 3 is formed on the support film 2. Specifically, a platinum (Pt) film, which forms the heater electrode 3 on the support film 2, is deposited in a thickness of 250 nm at 200° C. by using a vacuum evaporator.

A titanium layer (not illustrated) constituting an adhering layer for promoting adherence between the support film 2 and the heater electrode 3, is deposited by about 5 nm between the platinum film and the support film 2. The heater electrode 3 is formed by patterning by etching.

Next, the lower, or first, electric insulating layer 4 is formed by an LP-CVD process or a plasma CVD process on the heater electrode 3 and the support film 2 to cover all of the surface of the heater electrode 3. When there are recesses and projections on the surface of the first insulating layer 4, the surface may be polished.

Step of FIG. 3B

Next, the detection electrodes 6a, 6b are formed on the first electric insulating layer 4. Specifically, first, a metal thin film is formed by depositing a metal for the detection electrodes 6a, 6b on the first insulating layer 4 by a vacuum evaporator.

The detection electrodes 6a, 6b can be prevented from being exfoliated by forming a layer of titanium, chromium, nickel or the like as an adhering layer (not illustrated) for promoting adherence between the first insulating layer 4 and the detection electrodes 6a, 6b. The detection electrodes 6a, 6b in the comb-like shape are formed by patterning the metal thin plate.

Thereafter, a further insulating layer 9a is formed on the first insulating layer 4 to cover the detection electrodes 6a, 6b.

Step of FIG. 3C

Next, the further insulating layer 9a is thinned and flattened until the surfaces of the detection electrodes 6a, 6b are exposed. Specifically, the surface of the further insulating layer 9a is polished by CMP or the like. Polishing is stopped at a time at which the surfaces of the detection electrodes 6a, 6b are exposed from the further insulating layer 9a. Thereafter, the surfaces of the detection electrodes 6a, 6b may be cleaned, which further flattens them. Thus, the further insulating layer 9a becomes the second, or upper, insulating layer 9.

The change in the resistance of the sensitive film 5 cannot be detected unless the detection electrodes 6a, 6b are brought into direct contact with the sensitive film 5. Therefore, it is necessary to fully expose the surfaces of the detection electrodes 6a, 6b.

Step of FIG. 4A

Next, the sensitive film 5 is formed on the flattened second insulating layer (upper insulating layer) 9 to cover the exposed detection electrodes 6a, 6b to electrically connect the detection electrodes 6a, 6b and the sensitive film 5.

Specifically, first, a thin film for constituting the sensitive film 5 is formed by using a method of sputtering, sintering or the like. Here, an amorphous layer of the thin film may be crystallized by annealing. When a thin film of about several nanometers is formed, the film may be formed by ALE (atomic layer growing method) or ion beam sputtering. Further, the thin film is patterned to the shape of the sensitive film 5 by etching.

Then, the electrode lead-out port 4a is formed by etching the first insulating layer 4 and the second insulating layer 9.

Step of FIG. 4B

Next, the heater pads 7a and 7b and the detection electrode pads 7c and 7d are formed. Specifically, after depositing, for example, gold on the flattened insulating layer 9 by a vacuum evaporator, the deposit is patterned to shapes of the respective pads 7a through 7d by etching. At this time, adhering layers (not illustrated) comprising chromium are formed between the detection electrodes 6a, 6b and the heater electrode 3 and the respective pads 7a through 7d to promote adherence.

As the respective pads 7a through 7d, Al, platinum or the like can be used other than gold. The adhering layer may be constituted by a material having ohmic contact with the heater electrode 3 and may be titanium, nickel or the like.

Step of FIG. 4C

A mask 11 having an opening 11a is located in correspondence with the sensitive film 5 on a lower face of the substrate 1. That is, the mask 11 is formed on the side opposite to the face on which the sensitive film 5 is located. Specifically, the mask 11 is formed by forming a silicon oxide film or a silicon nitride film at the lower face of the substrate 1 and then forming the opening 11a by etching or the like.

Thereafter, the hollow cavity 8 is formed at an area in correspondence with the opening 11a of the mask 11 by etching the substrate 1 using the mask 11. Specifically, the silicon (Si) that makes the substrate 1 is anisotropically etched by a TMAH solution or a KOH solution from the rear face of the substrate 1, which is the lower face in the figures.

When etching is carried out by the TMAH solution, a protection film may be provided such that the surface of the pads 7a through 7d, the sensitive film 5 and the like formed on the front, or upper, side of the substrate 1 are not etched. Also jig may be used such that a portion dipped into the TMAH solution is only the face to be etched. Thus, the illustrated gas sensor is completed.

Accordingly, due to the way the flattened insulating layer 9 fills the surroundings of the detection electrodes 6a, 6b, steps, or differences in elevation, formed by the detection electrodes 6a, 6b are reduced, and the sensitive film 5 can be formed on the flattened face. This inhibits breakage of the sensitive film 5 and improves the reliability of the gas sensor.

Although the surfaces of the detection electrodes 6a, 6b and the surface of the flattened insulating layer 9 are preferably flush, they may be non-flush. If the surfaces are not flush, the maximum difference between the two surfaces, as measured in a direction perpendicular to the plane of the sensitive film 5, should be smaller than the film thickness of the sensitive film 5, to inhibit breakage of the sensitive film 5. That is, the maximum difference between a high point and a low point, or the step elevation, at the face in contact with the sensitive film 5 should be smaller than the film thickness of the sensitive film 5.

When the surfaces of the detection electrodes 6a, 6b are covered by the further insulating layer 9a, the detection electrodes 6a, 6b are exposed by polishing the second electrically insulating layer 9a. Therefore, the faces of the detection electrodes 6a, 6b that are to contact the sensitive film 5 can be flattened.

Since the substrate 1 is provided with the hollow cavity 8, heat transfer to the substrate 1 is relatively impeded. Therefore, the temperature of the sensitive film 5 is more easily be elevated and power consumption is reduced.

### Second Embodiment

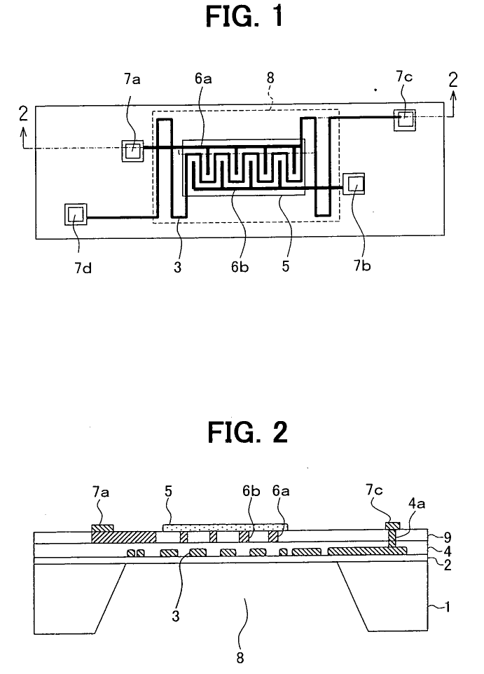

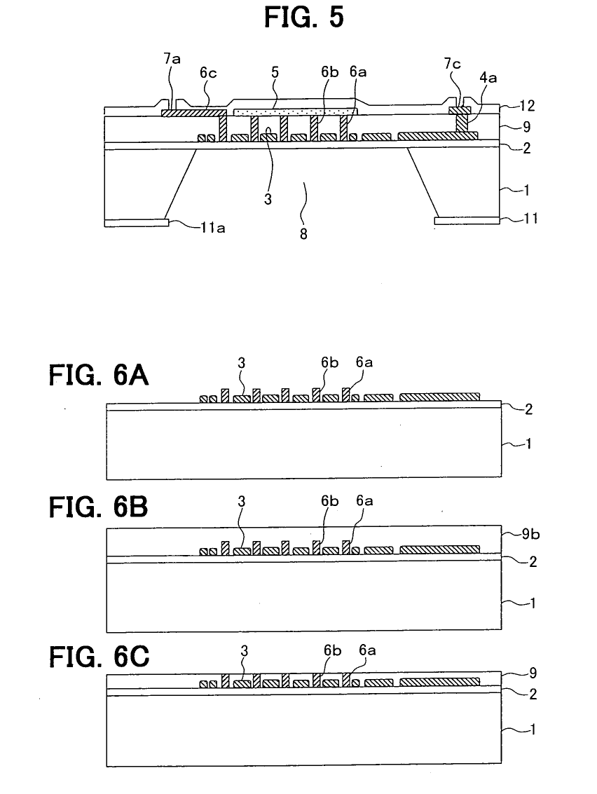

The second embodiment differs from the first embodiment in that the detection electrodes 6a, 6b are formed on a face the same as that of the heater electrode 3. A plan view of a gas sensor according to the second embodiment is omitted since the plan view is the same as FIG. 1. However, FIG. 5 shows a cross sectional view of the gas sensor according to the second embodiment taken along the section 2-2 of FIG. 1. The following explanation will mainly be given of parts that differ from the first embodiment, and parts of FIG. 5 that are the same as those of FIG. 2 are given the same reference number and are not explained.

As shown by FIG. 5, the heater electrode 3 is formed on the support film 2 and the detection electrodes 6a, 6b are formed on the same face that the heater electrode 3 is on. The meandering heater electrode 3 and the detection electrodes 6a, 6b are alternately arranged with predetermined intervals as shown in FIG. 1, and the heater electrode 3 and the detection electrodes 6a, 6b are electrically insulated from each other. The film thickness of the detection electrodes 6a, 6b is greater than that of the heater electrode 3 as shown in FIG. 5.

The flattened insulating layer 9 is formed on the support film 2 and the heater electrode 3, and the flattened insulating layer 9 is flattened at its surface along with the surfaces of the detection electrodes 6a, 6b. the flattening exposes the surfaces of the detection electrodes 6a, 6b while leaving the heater electrode 3 covered.

That is, the surroundings of the detection electrodes 6a, 6b are filled, and the surfaces of the detection electrodes 6a, 6b and the surface of the flattened insulating layer 9 are substantially flush.

At the end portions of the detection electrodes 6a, 6b, wirings 6c are formed in a linear shape at the surface of the flattened insulating layer 9 to electrically connect to exposed end surfaces of the detection electrodes 6a, 6b as shown in FIG. 5. The outer ends of the wirings 6c are electrically connected to the electrode pads 7a, 7b.

Thus, at locations of the plan view at which the detection electrodes 6a, 6b and the heater electrode 3 seem to intersect, the detection electrodes 6a, 6b are spaced from the heater electrode 3 and electrically insulated from the heater electrode 3 by the flattened insulating layer 9.

In the second embodiment the heater electrode 3 and the detection electrodes 6a, 6b are formed on the same face. Therefore, the electric insulating layer 4, which is employed in the first embodiment, is omitted.

A filter 12 for permitting passage of only a specific gas is formed on the sensitive film 5. Thus, the selectivity of the sensor is improved. In this case, for example, in order to improve the selectivity of hydrogen, a silicon oxide film may be employed as the filter 12. Although the silicon oxide film permits passage of hydrogen having a small molecular size, a molecule having a larger molecular size cannot permeate the filter 12. Therefore, only hydrogen gas can reach the electric film 5. Thus, only hydrogen gas can be detected.

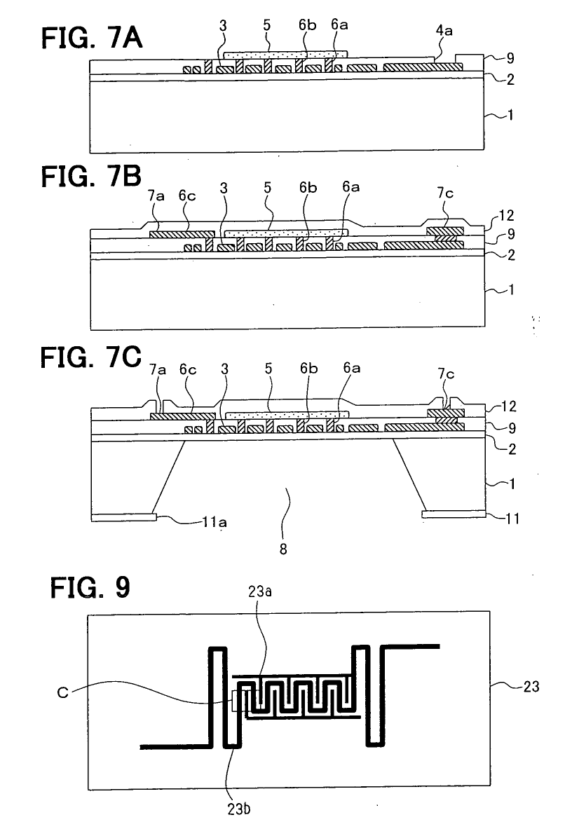

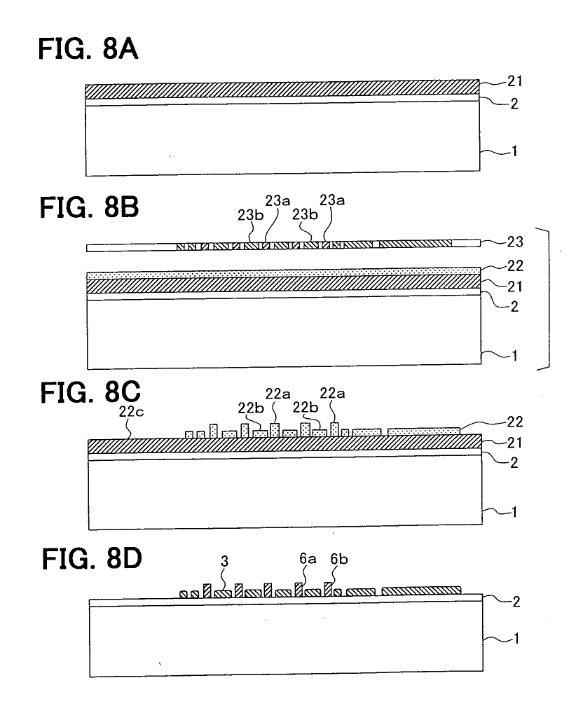

Next, an explanation will be given of a method of fabricating the gas sensor of the second embodiment with reference to FIGS. 7A, 7B, 7C, 8A, 8B, 8C, 8D, 9, 10A, 10B, and 10C. (Note that the stages of FIGS. 8A, 8B, and 8C are prior to the stages of FIGS. 7A, 7B, and 7C.) The following description will mainly cover parts of the method of fabricating the gas sensor that are different from the steps of the first embodiment.

Step of FIG. 6A

After forming the support film 2 on the substrate 1, the heater electrode 3 and the detection electrodes 6a, 6b are simultaneously formed on the support film 2 at different thicknesses. Specifically, first, after depositing a titanium layer (not illustrated) on the support film 2, which forms an adhering layer for adhering the heater electrode 3, the detection electrodes 6a, 6b to the support film 2, a metal thin film, which is platinum, for making the heater electrode 3 and the detection electrodes 6a, 6b, is deposited by a vacuum evaporator. The platinum film is patterned to the shapes of the heater electrode 3 and the detection electrodes 6a, 6b by etching or the like.

A detailed explanation will be given of the steps of forming the heater layer and the detection electrodes with reference to FIGS. 8A, 8B, 8C, which are step views that correspond to FIGS. 6A, 6B and 6C.

First, as shown by FIG. 8A, a platinum film 21, which is 250 nm or more in thickness, is deposited on the support film 2. Next, as shown by FIG. 8B, a positive type photoresist 22 is coated on the platinum film 21 by spin coating or the like. The photoresist 22 is developed by using a photo mask 23 formed with a pattern 23b of the heater electrode 3 and patterns 23a of the detection electrodes 6a, 6b.

Thus, as shown by FIG. 8C, in the photoresist 22, the thickness of a heater electrode portion 22b, which corresponds with the heater electrode 3, is thinner than detection electrode portions 22a, which correspond with the detection electrodes 6a, 6b. An explanation will follow of a method, which is used in this embodiment, of forming patterns having different thicknesses to the photoresist 22 by one-time development.

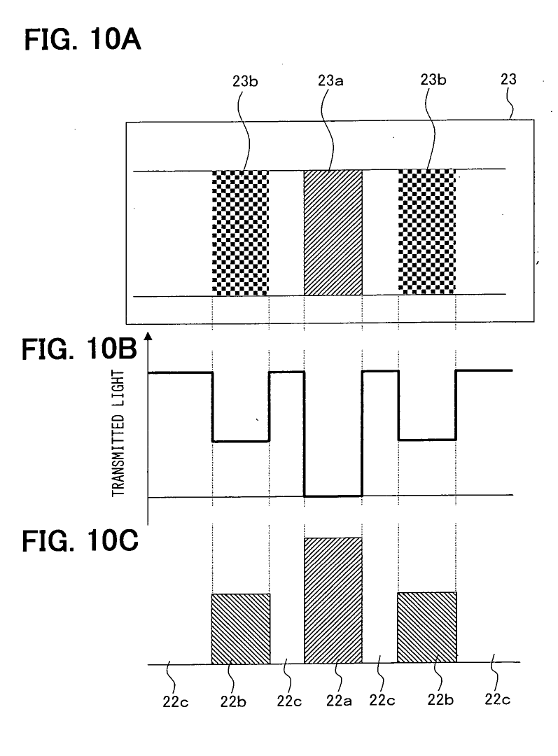

FIG. 9 is a plan view of the photo mask 23. FIG. 10A is an enlarged plan view of window C in FIG. 9. FIG. 10B graphically shows amounts of transmitted light at parts of the photoresist 22 when light is irradiated through the photo mask 23 of FIG. 10A. FIG. 10C is the cross sectional shape of the photoresist 22 after being developed by the light indicated in FIG. 10B.

As shown by FIG. 9, at the portions of the photo mask 23 that correspond with the detection electrodes 6a, 6b, patterns 23a that completely block light, with chromium or the like, are formed. At a portion that corresponds with the heater electrode 3, a fine pattern 23b, the resolution of which is equal to or smaller than the resolution of the exposure apparatus, is formed.

As shown by FIG. 10A, the fine pattern 23b is formed with a number of very small rectangular windows for transmitting light, and the windows are formed to distribute a predetermined density of light. The dimension of the rectangular windows is equal to or smaller than the resolution of the exposure apparatus used for exposing the photo mask 23. For example, in the case in which an exposure apparatus used is a 10 to 1 contraction exposure apparatus, when the resolution is 1 micrometer, the size of a side of each rectangular window is equal to or smaller than 1 micrometer by using a reticle size that is ten times the size of the window.

At other portions in the photoresist, light is completely transmitted.

When light is irradiated to the photoresist 22 through the photo mask 23, as indicated by FIG. 10B, the level of light transmission at a portion that corresponds with the light blocking pattern 23a becomes 0%. The level of light transmission corresponding to a portion that is not part of the pattern is 100%, and the level of light transmission a portion that corresponds with the fine pattern 23b is between 0% and 100%. The level of light transmission at the fine pattern 23b can be changed by varying the density of the rectangular windows.

When the photoresist 22 is developed, as shown by FIG. 10C, the photoresist portion 22a, which corresponds with the light blocking pattern 23a, is unaffected and thus has the greatest thickness. The thickness of the photoresist portion 22b, which corresponds with the fine pattern 23b, is reduced. At other photoresist portions 22c, the photoresist is completely removed. Therefore, the photoresist is shaped as shown by FIG. 8C.

The platinum film is then etched using the photoresist 22, which is formed with the patterns of varying thickness. The etching is dry etching, and the preferred etching gas is Argon gas or CF₄ gas, which is for etching the metal, added to O₂ gas, which is for etching the photoresist 22.

When the flow rates or pressures of the respective gases are set such that the rate of etching of the platinum film by the argon gas or the CF₄ gas and the rate of etching of the photoresist 22 are equal, the shape of the patterned photoresist 22 is transferred to the platinum film 21 as it is. As a result, as shown by FIG. 8D, the thickness of the heater electrode 3 can be made less than that of the detection electrodes 6a, 6b.

Step of FIG. 6B

Next, an electrically insulating layer 9b is formed on the support film 2 to cover the heater electrode 3 and the detection electrodes 6a, 6b.

Step of FIG. 6C

The electric insulating layer 9b is machined away until the surfaces of the detection electrodes 6a, 6b are exposed. That is, a flattening step, like that for thinning the second electrically insulating layer 9a in the first embodiment (step of FIG. 3C), is performed. As a result, the electrically insulating layer 9b becomes the flattened insulating layer 9.

Step of FIG. 7A

The sensitive film 5 and the electrode leadout ports 4a are formed.

Step of FIG. 7B

After forming the pads, a silicon oxide film for making the filter 12 is formed on the flattened insulating layer 9 and on the sensitive film 5 and the respective pads 7a through 7d.

Step of FIG. 7C

After forming the hollow cavity, parts of the filter 12 that are above the respective pads 7a through 7d are removed by etching or the like. The respective pads 7a through 7d are electrically connected to circuits by bonding wires or the like. The gas sensor of the second embodiment is thus finished.

In the gas sensor of the second embodiment, the sensitive film 5 is formed on a flat face and, therefore, breakage of the sensitive film 5 is inhibited, which improves the reliability of the gas sensor.

Since the heater electrode 3 and the detection electrodes 6a, 6b are formed on the same face, it is not necessary to provide an electrically insulating layer between the heater electrode 3 and the detection electrodes 6a, 6b. The heater electrode 3 and the detection electrodes 6a, 6b can be formed simultaneously. Therefore, in this embodiment, the number of fabricating steps is relatively low.

The hollow cavity forming step is carried out after covering the sensitive film 5, the wiring 6c and the respective pads 7a to 7d by the filter 12. Therefore, the sensitive film 5, the wiring 6 and the respective pads 7a through 7d are protected against the etching solution of TMAH solution or the like in the hollow cavity forming step.

The filter 12 prevents deterioration of the sensitive film 5 and the detection electrodes 6a, 6b by miscellaneous gases present in a surrounding atmosphere and prevents dirt or the like from adhering to the sensitive film 5 and the detection electrodes 6a, 6b.

### Third Embodiment

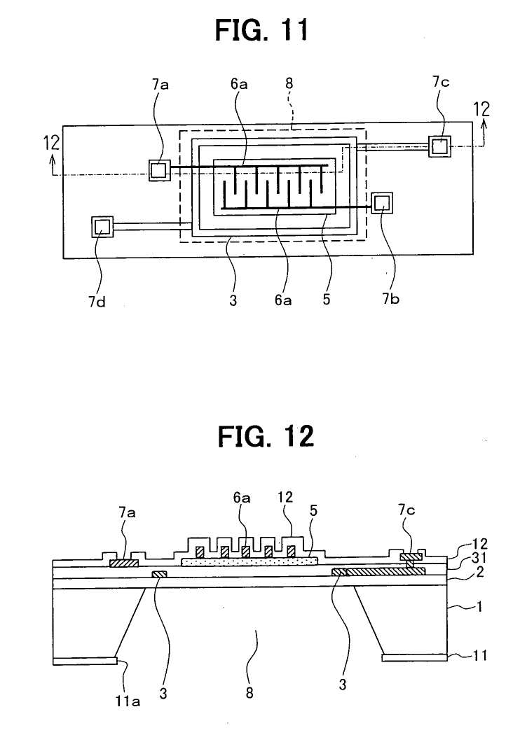

The third embodiment differs from the first and the second embodiments in that the detection electrodes 6a, 6b are formed on the sensitive film 5 and the heater electrode 3 is not located directly below the sensitive film 5. The third embodiment is described with particular reference to FIGS. 11 and 12. The following explanation will mainly cover parts that differ from the first and the second embodiments, and parts of FIGS. 11 and 12 that are the same as corresponding parts of FIGS. 1 and 2 have the same reference numbers and will not be described again.

As shown by FIG. 12, the heater electrode 3 is formed on the support film 2. The heater electrode is formed below and surrounding the area that is directly below the sensitive film 5, as shown by FIG. 11. That is, the heater electrode 3 is located outside of an imaginary projection of the sensitive film 5 that extends in the normal direction of the sensitive film 5. In other words, the heater electrode 3 is located outside of the perimeter of (or a projection of the perimeter of) the sensitive film 5. Specifically, the heater electrode 3 has a frame shape.

The heater electrode 3 is arranged between the outer periphery of the hollow cavity 8 (at the surface of the substrate 1) and the outer periphery of the sensitive film 5. When viewed from above, the sensitive film 5, the outer periphery of the hollow cavity 8 (at the surface of the substrate 1), and the outer periphery of the heater electrode 3 have similar shapes.

The hollow cavity 8 and the heater electrode 3 and the sensitive film 5 are arranged such that, for example, the area surrounded by the outer periphery of the heater electrode 3 is about 80% of the area surrounded by the outer periphery of the hollow cavity 8 (at the surface of the substrate 1) and the area surrounded by the outer periphery of the sensitive film 5 is about 80% of the area surrounded by the outer periphery of the heater electrode 3.

An electrically insulating layer 31 is formed on the support film 2 and the heater electrode 3. The sensitive film 5 is formed flatly on a portion of the insulating layer 31 surrounded by the heater electrode 3 and not directly above the heater electrode 3.

The detection electrodes 6a, 6b are formed on the sensitive film 5. The heater pads 7c and 7d and the electrode pads 7a and 7b are formed on the electrically insulating layer 31. The filter 12 is formed on the insulating layer 31, the sensitive film 5, the detection electrodes 6a, 6b and the respective pads 7a through 7d. Further, the filter 12 is perforated above the respective pads 7a through 7d.

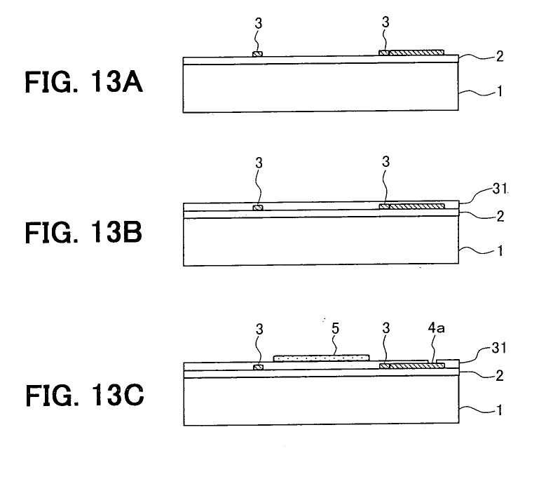

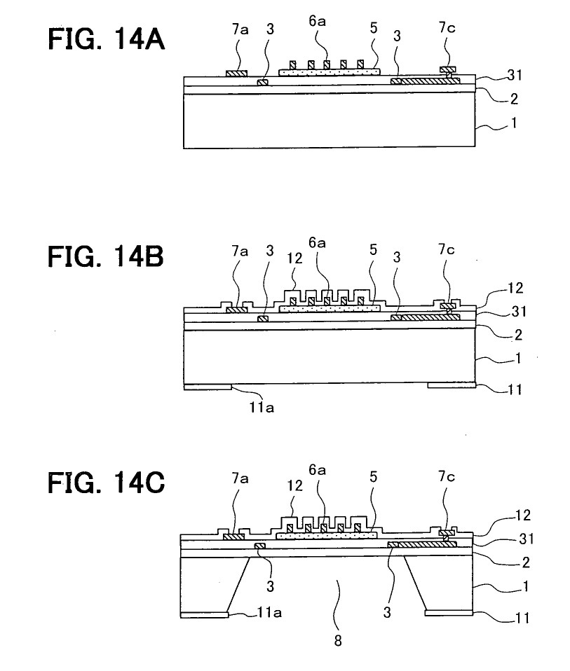

A description of the method of fabricating the gas sensor according to this embodiment will follow with reference to FIGS. 13A, 13B, 13C, 14A, 14B and 14C. The description will focus on parts that differ from the preceding embodiments.

Step of FIG. 13A

First, the support film 2 is formed on the substrate 1. Thereafter, a heater layer is formed.

Step of FIG. 13B

There is carried out a step of forming an electrically insulating layer for forming the electrically insulating layer 31 on the heater electrode 3.

Step of FIG. 13C

The photosensitive film is applied and the electrode lead-out ports 4a are formed.

Step of FIG. 14A

The detection electrodes are formed. The pads are formed simultaneously with the detection electrodes. That is, after depositing a gold film on the insulating layer 31 and the sensitive film 5 by a vacuum evaporator, the gold film is patterned in the shapes of the detection electrodes 6a, 6b and the pads 7a through 7d by etching.

A chromium adhering layer (not illustrated) is deposited between the detection electrodes 6a, 6b and the sensitive film 5.

Step of FIG. 14B

The filter 12 is formed. Also, a mask is formed.

Step of FIG. 14C

The hollow cavity is formed. Thereafter, the parts of the filter 12 that correspond to the respective pads 7a to 7d are removed. This completes the gas sensor.

In this embodiment, the detection electrodes 6a, 6b are formed on the sensitive film 5, the heater electrode 3 is formed around but not directly below the sensitive film 5. Accordingly, the sensitive film 5 is formed on a flat face that has no recesses and projections. Therefore, breakage of the sensitive film 5 is inhibited, which improves the reliability of the gas sensor.

Particularly, when the sensitive film 5 is thinner than the detection electrodes 6a, 6b, it is advantageous to provide the detection electrodes 6a, 6b on the sensitive film 5 as in this embodiment. This is because when the detection electrodes 6a, 6b are formed below the sensitive film 5, there is a greater possibility of breaking the sensitive film 5 due to steps, or height differences, created by the detection electrodes 6a, 6b.

Since the heater electrode 3 is provided between the outer periphery of the hollow cavity 8 and the outer periphery of the sensitive film 5, the sensitive film 5 can be heated from its surroundings without being heated from directly below. Since the heater electrode 3 is formed above the hollow cavity 8, heat generated from the heater electrode 3 is impeded from escaping to the substrate 1.

Generally, lines of equal temperature, or isotherms, in the sensitive film 5 are dependent upon the shapes of the hollow cavity 8, the heater electrode 3 and the sensitive film 5. Therefore, by shaping the outer peripheries of the hollow cavity 8, the heater electrode 3 and the sensitive film 5 as shown in this embodiment, the isotherms at the support film 2 and the sensitive film 5 above the hollow cavity 8 can be made concentric and accordingly, temperature control of the sensitive film 5 can be performed easily.

### Fourth Embodiment

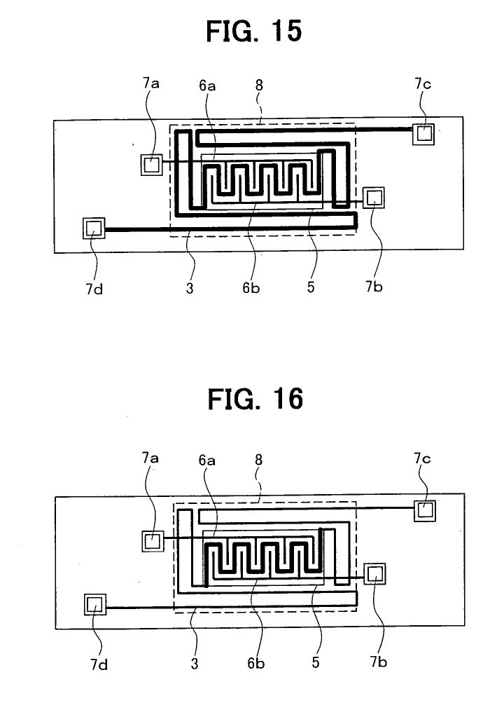

The heater electrode 3 need not only be arranged directly below the sensitive film 5 to totally heat the sensitive film 5 but may meander over the hollow cavity 8 and above the support film 2 as shown by FIG. 15.

By such a construction that is capable of heating the surroundings of the sensitive film 5, heat transfer from the sensitive film 5 is reduced. Since all of the films above the hollow cavity 8 can be heated uniformly, deviation in the temperature distribution at the sensitive film 5 is reduced and the detection sensitivity is more constant.

### Fifth Embodiment

As shown by FIG. 16, the width of a part of the heater electrode 3 located directly below the sensitive film 5 may be increased. Thus, temperature control of the sensitive film 5 can easily be carried out by limiting abrupt heat generation of the heater electrode 3. The heat generation at locations surrounding the sensitive film 5 is increased compared to parts directly below the sensitive film by relatively reducing the width of the heater electrode 3 at locations not directly below the sensitive film 5. This improves the temperature uniformity of the sensitive film 5.

### Sixth Embodiment

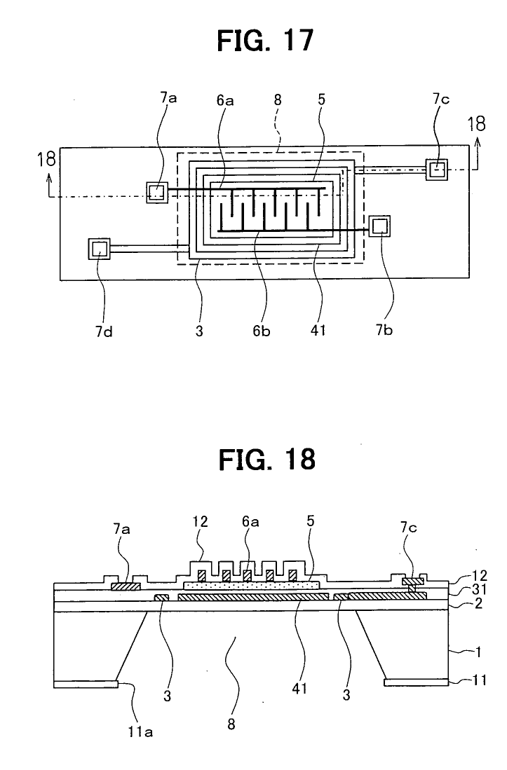

As shown by FIG. 17 and FIG. 18, the third embodiment may be modified by forming a temperature control film 41 on the inner side of the heater electrode 3 on the same level, or face, as that of the heater electrode 3. The temperature control film 41 is for facilitating heat transfer from the heater electrode 3.

As shown by FIG. 17, when viewed from above the sensitive film 5, the outer periphery of the temperature control film 41 is located between the inner periphery of the heater electrode 3 and the outer periphery of the sensitive film 5. The temperature control film 41 is constituted by a flattened film having a uniform thickness such that recesses and projections are not formed at the face on which the sensitive film 5 is formed. That is, the temperature control film 41 is constituted by a solid film larger than the sensitive film 5 in area and smaller than the area encompassed by the heater electrode 3.

A material that is the same as that of the heater electrode 3 can be used for the temperature control film 41. The temperature control film 41 can be formed simultaneously with the heater electrode 3 by changing the pattern in the step of forming the heater layer.

By providing the temperature control film 41 in this way, the temperature uniformity of the sensitive film 5 is improved. As a result, the sensitivity is improved. Since the temperature control film 41 is larger than the sensitive film 5, the sensitive film 5 is formed on a flat face, the sensitive film 5 is inhibited from being broken and, at the same time, the temperature uniformity of the sensitive film 5 is improved.

### Seventh Embodiment

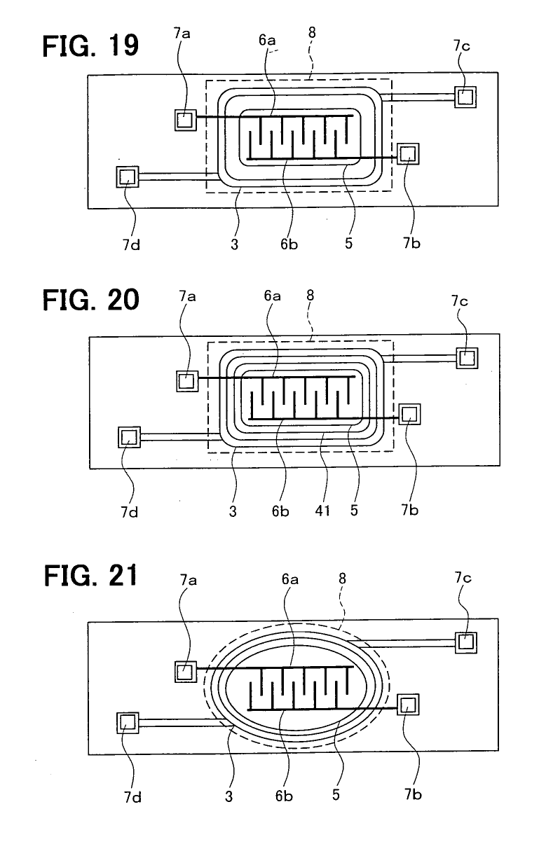

As shown by FIG. 19 and FIG. 20, the third embodiment may be modified by chamfering or rounding a corner portion of the heater electrode 3. Also, a corner portion of the sensitive film 5 may be chamfered or rounded. The sixth embodiment may be modified by chamfering or rounding a corner portion of the temperature control film 41.

Generally, it is normal that isotherms on the substrate 1, resulting from heat generation of the heater electrode 3, do not have angles and have rounded corners. Therefore, when the corner portions of the heater electrode 3, the sensitive film 5 or the temperature control film 41 are chamfered or rounded, the shape of the heater electrode 3, the sensitive film 5 or the temperature control film 41 is matched more closely to the shape of the isotherms, and therefore, temperature control is more easily accomplished. Further, the deviation of the temperature distribution of the sensitive film 5 is reduced.

### Eighth Embodiment

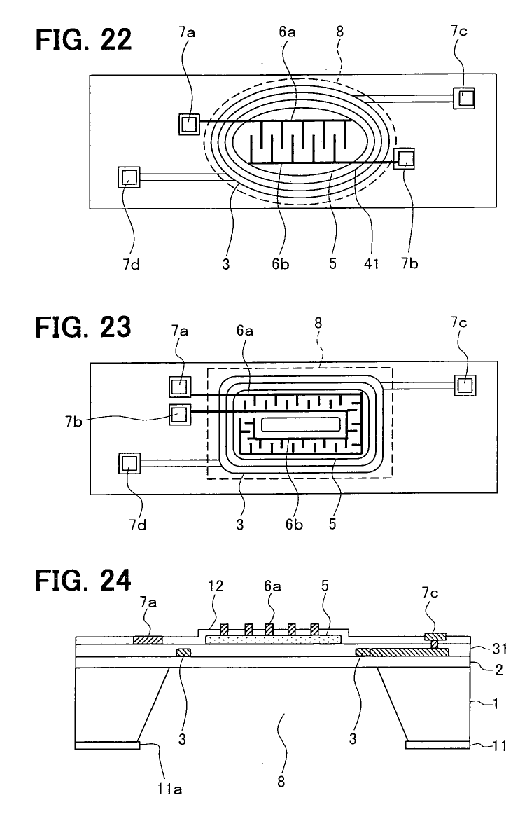

As shown by FIG. 21 and FIG. 22, the third embodiment may be modified by forming the path of the heater electrode 3, the sensitive film 5 or the temperature control film 41 in an elliptical shape. Thus, the deviation of the temperature distribution of the sensitive film 5 can further be reduced compared to the seventh embodiment.

### Ninth Embodiment

As shown by FIG. 23, the third embodiment can be modified by forming the sensitive film 5 in an oval shape. Generally, the temperature distribution in the sensitive film 5 depends on the distance from the heater electrode 3. Therefore, by forming the sensitive film 5 in an oval shape, the sensitive film 5 can be arranged at a constant distance from the heater electrode 3 by eliminating a part of the heater electrode 3 that is remote from the sensitive film 5. This reduces the deviation of the temperature distribution in the sensitive film 5.

### Tenth Embodiment

The third embodiment can be modified by exposing the detection electrode 6a, 6b from the filter 12 as shown by, for example, FIG. 24. However, since the sensitive film 5 and the detection electrodes 6a, 6b must be connected electrically, electric connection with the detection electrodes 6a, 6b is ensured by forming contact holes in, for example, the filter 12.

### Eleventh Embodiment

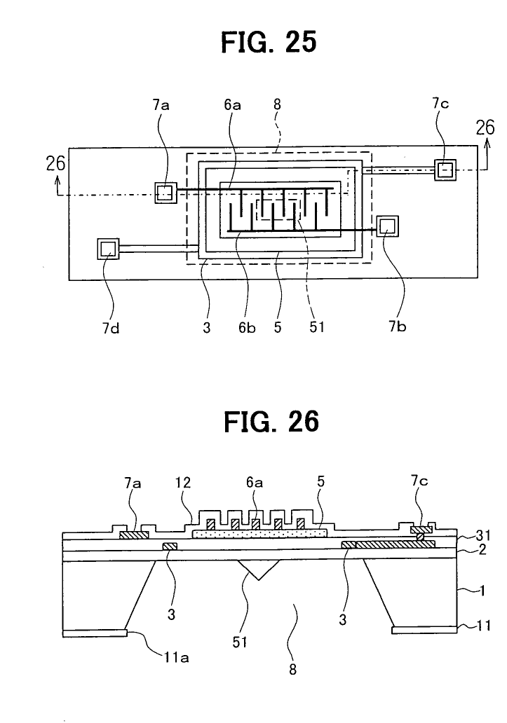

The first to third embodiments can be modified, as shown by FIG. 25 and FIG. 26, by forming a projection 51 in the support film 2 on the side of the hollow cavity 8. The projection 51 is provided at a location of the support film 2 at which the temperature is likely to be high, to make temperature of the sensitive film 5 more uniform.

Specifically, the projection 51 is arranged at a central portion of the sensitive film 5 with a shape similar to that of the sensitive film 5 when viewed from above the sensitive film 5. It is preferred to provide the projection 51 with an area of about 10 to 50% of the area of the sensitive film 5. The projection 51 can be formed by, for example, leaving a portion of the substrate 1.

Thus, heat at a location at which the temperature is likely to be high is transferred to the projection 51 and the temperature of the sensitive film 5 directly above the projection 51 is lowered accordingly. Therefore, temperature control of the sensitive film 5 is more easily accomplished by reducing the deviation of the temperature distribution in the sensitive film 5.

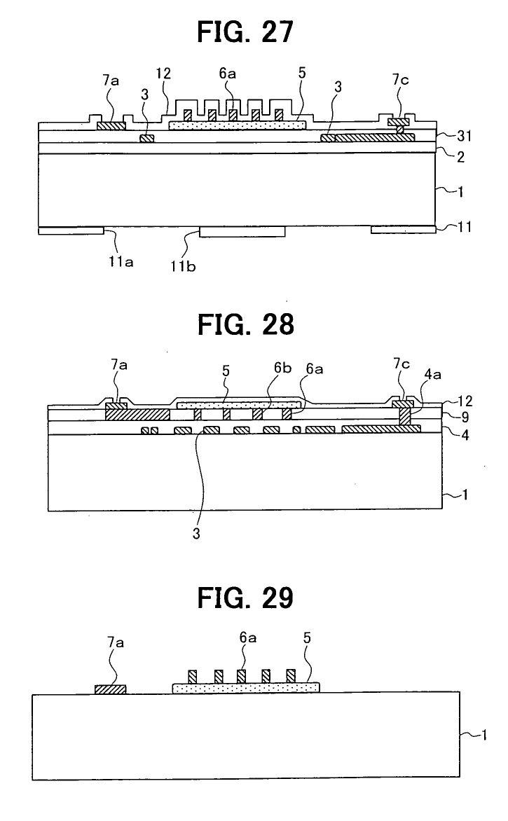

When such a projection 51 is formed, a part of the opening 11a is masked by a mask 11b. That is, as shown by FIG. 27, the mask 11b is formed below the sensitive film 5. The mask 11b corresponds to the projection 51 and partially covers the opening 11a.

In the step of forming the hollow cavity, the projection 51 can be formed by etching the substrate 1 via the mask 11 in a manner similar to the methods of the above-described embodiments while leaving a portion of a bottom face of the hollow cavity 8.

In this way, the projection 51 can be formed by a portion of silicon (substrate) that remains after the anisotropic etching, and therefore, the projection 51 can be formed without adding a step.

### Twelfth Embodiment

The first through the third embodiments can be modified, as shown by FIG. 28. That is, when the electrically insulating substrate 1 is an alumina substrate, a sapphire substrate or the like, the heater electrode 3, the temperature control film 41 and the like may be formed directly on the substrate 1 without forming the support film 2. Such an electrically insulating substrate frequently has favorable thermal insulating characteristics, and therefore, heat is relatively impeded from transferring from the heater electrode 3. Therefore, the hollow cavity 8 can be omitted.

### Thirteenth Embodiment

In the twelfth embodiment, when gases can be sensed at room temperature, the heater electrode 3 may be omitted. That is, as shown by FIG. 29, in a gas sensor capable of detecting a gas at room temperature, the sensitive film 5, which is sensitive to the gas to be detected, may be formed on the electrically insulating substrate 1 and the detection electrodes 6a, 6b may be formed on the sensitive film 5.

In such a gas sensor for detecting gas at room temperature, the sensitive film 5 can be formed at a face on the substrate that has no recesses and projections, and therefore, the sensitive film 5 is inhibited from being broken.

### Fourteenth Embodiment

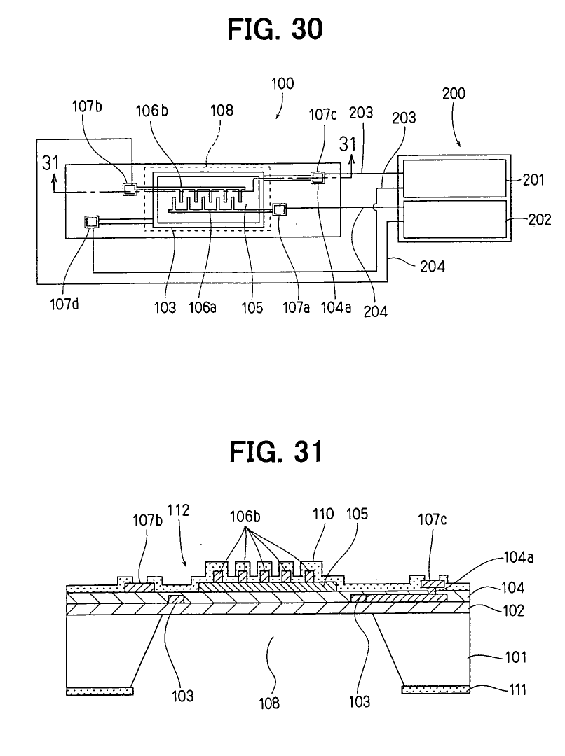

FIG. 30 and FIG. 31 show a gas sensor that includes a sensing element 100 and circuit 200. A support film 102 is formed on a surface of a substrate 101, which is, for example, silicon. The support film 102 is a laminated composite film formed with a silicon oxide film and a silicon nitride film and is formed to provide tensile stress. In detail, the silicon oxide film is provided with compressive stress, the silicon nitride film is provided with tensile stress, and the net stress in the support film 102 is a weak tensile stress, which is determined by adjusting film thicknesses of the silicon oxide film and the silicon nitride film.

There is formed a heater 103 for controlling temperature of a sensitive film 105 on the support film 102. The heater 103 is formed in a frame shape at a central portion of the substrate 101, and the heater 103, which has a linear shape, is extended from two corners of the frame shape in the directions of respective ends of the substrate 101. The heater 103 is made by a material that includes a noble metal substance of platinum, gold or the like, RuO₂, polysilicon or the like.

An electrically insulating film 104 is formed on the heater 103. The electrically insulating film 104 includes a film combined with a silicon oxide film and a silicon nitride film. Ideally, the support film 102 and the electrically insulating film 104 are symmetric, and the heater 103 is between them. This is for the following reason.

The support film 102 and the insulating film 104 are arranged above and span a hollow cavity 108 formed in the substrate 101. Therefore, by warming the support film 102 and the electrically insulating film 104 with the heater 103, the support film 102 or the electrically insulating film 104 is bent by a difference in the thermal expansion coefficients of the silicon oxide film and the silicon nitride film. However, the bending can be inhibited when the support film 102 and the electrically insulating film 104 are formed symmetrically.

A linear electrode lead-out port 104a is formed above an end of the heater 103 in the linear shape in the insulating film 104, and the end portion of the heater 103 is exposed.

A physical value of the sensitive film 105 is changed by the adsorption and desorption of gases. The sensitive film 105 is located on an inner side of the heater 103, which is frame-shaped when viewed from above. Therefore, the heater 103 is not arranged directly below the sensitive film 105.

The sensitive film 105 can be made of an oxide semiconductor of SnO₂, TiO₂, ZnO, In₂O₃ or the like. A physical value of the sensitive film 105, for example, electric resistance (hereinafter, simply referred to as resistance) or the like is changed by the presence or absence of gases. The following is an explanation of the detection of changes in the resistance.

The sensitive film 105 may have a thickness equal to or smaller than 10 nm. By sizing the sensitive film 105 in this way, the response is improved by reducing the time period for diffusion of gas by preventing gas from being diffused to an inner portion of the sensitive film 105 and by making the gas react with the surface of the sensitive film 105. The resistance of the sensitive film 105 is changed by forming a depletion layer by adsorbing the gas and therefore, a large sensitivity is provided by setting the sensitive film 105 to a film thickness that is the same as the thickness of the depletion layer. Further, depending on the gas, sensitivity to the gas may be improved by adding an impurity to the sensitive film 105.

Detection electrode 106a, 106b are on the sensitive film and detect the change in the resistance of the sensitive film 105. A pair of the detection electrodes 106a and 106b is provided, and the respective detection electrodes 106a and 106b are formed in a comb-like shape. Further, ends of the detection electrodes 106a and 106b extend above the substrate 101, and pads 107a and 107b for the detection electrodes are formed at the end portions of each of the detection electrodes 106a and 106b.

The detection electrodes 106a and 106b can be made of a noble metal such platinum, gold or the like, or aluminum or the like. The pads 107a and 107b can be made of aluminum, gold or the like. As discussed later a material that facilitates adherence to bonding wires is formed at the pads 107a and 107b for the detection electrodes.

When the sensitive film 105 is thinner than the detection electrodes 106a, 106b, it is preferred to provide the detection electrodes 106a and 106b on the sensitive film 105 in this way. This is because, if the sensitive film 105 is thinner than the detection electrodes 106a and 106b, when the detection electrodes 106a and 106b are located below the sensitive film 105, the possibility of breaking the photosensitive film 105 due to steps, or differences in height of the detection electrodes 106a and 106b, increases.

Pad portions 107a through 107d for the heater are electrically connected to the heater 103 above the electrode lead-out ports 104a.

A filter 110 is formed on the detection electrodes 106a and 106b, the sensitive film 105 and the respective pads 107a to 107d. The filter 110 permits only a specific gas to pass and thus improves the gas selectivity of the sensor. For example, an oxide film is formed as the filter 110 to promote selectivity of hydrogen gas.

In this case, the sensitive film 105 and the detection electrodes 106a and 106b are covered by the oxide film filter 110, and deterioration of the sensitive film 105 and the detection electrodes 106a and 106b by miscellaneous gases in a surrounding atmosphere is prevented, and dirt or the like is prevented from adhering to the sensitive film 105 and the detection electrodes 106a and 106b. Also, the filter protects the sensitive film 105 and the detection electrodes 106a and 106b or the like from etching solution when the hollow cavity 108 is formed in the substrate 101.

Parts of the filter 110 above the respective pads 107a to 107d are perforated, and the respective pads 107a through 107d are exposed.

The hollow cavity is formed in the lower face of the substrate 101 below the heater 103. Thus, the support film 102, the insulating film 104, the heater 4 and the sensitive film 105 are thin-walled parts formed at locations corresponding to the hollow cavity 108. Hereinafter, a thin-walled part formed above the hollow cavity 108 is referred to as membrane and a part of the membrane on which the heater 103 and the sensitive film 105 or the like is formed is referred to as a thin-walled detecting portion 112.

Since the heater 103 is arranged above the hollow cavity 108 in this way, heat from the heater 103 is impeded from being carried away by the substrate 101. Accordingly, the thermal insulation characteristics are favorable. Therefore, power consumption is reduced by limiting heat transfer from the heater 103.

For example, when the membrane has a rectangular shape, a side of which is about 1 mm, and the thickness of the membrane is equal to or smaller than several micrometers, the temperature of the sensitive film 105 can be elevated to several hundred degrees in 10 msec or less.

At a location of the lower face of the substrate 101 surrounding the opening of the hollow cavity 108, a mask film 111 of an oxide film or the like is formed.

The circuit 200 includes a heater circuit control circuit 201, for controlling the temperature of the heater 103, and a sensitive film change analyzing circuit 202, for analyzing changes in the resistance of the sensitive film 105.

The heater temperature control circuit 201 is electrically connected to the pads 107c and 107d for the heater by wirings 203. The sensitive film change analyzing circuit 202 is electrically connected to the pads 107a and 107b for the detection electrodes by wirings 204. The electric connection can be accomplished by, for example, bonding wires.

The heater temperature control circuit 201 and the sensitive film change analyzing circuit 202 are connected. Thus, the temperature of the heater 103 can be controlled to reach a desired temperature by the heater temperature control circuit 201, and when the heater 103 reaches a desired temperature or when a desired time period has elapsed, a change in the resistance of the sensitive film 105 can be inputted as a signal to the analyzing circuit 202.

Next, a method of fabricating such a gas sensor is described. First, the substrate 101 is prepared and the support film 102 is formed. After forming the heater 103 by forming a platinum film or the like on the support film 102 and after patterning the film, the electrically insulating film 104 is formed and the electrode lead-out ports 104a are formed. Next, after forming the sensitive film 105, the detection electrodes 106a and 106b and the respective pads 107a through 107d are formed by forming an aluminum film or the like and by patterning the film.

Subsequently, the filter 110 is formed on the electrically insulating film 104, the sensitive film 105 and the detection electrodes 106a and 106b. Then, the mask film 111 is formed at the lower face of the substrate 101. Further, the hollow cavity 108 is formed by anisotropically etching the substrate 101 by a TMAH solution or the like while masking with the mask film 111. Thereafter, the respective pads 107a through 107d and the heater temperature control circuit 201 and the change analyzing circuit 202 are electrically connected by wire bonding or the like.

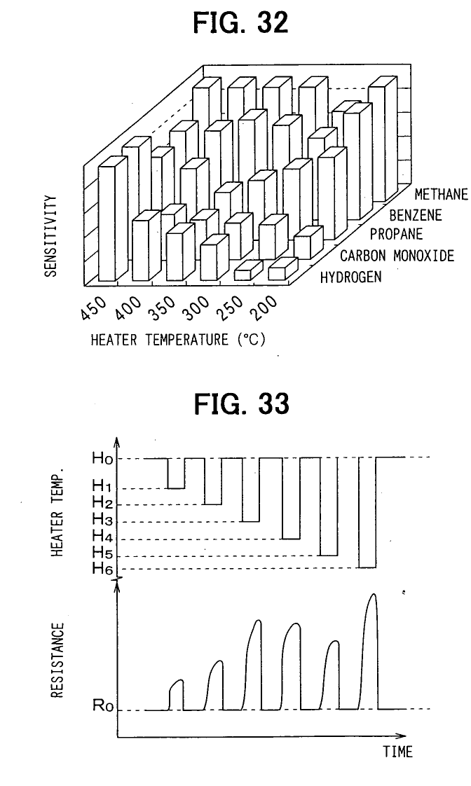

Next, a method of detecting a gas by using the gas sensor will be described. Several kinds of gases are identified, and their concentrations are identified. FIG. 32 shows the sensitivity of the sensitive film 105 (change in the resistance) when the sensitive film 105 at various temperatures between 200° C. and 450° C. is exposed to an atmosphere of various gases (hydrogen, carbon monoxide and the like) having the same concentration.

As shown by FIG. 32, the sensitivities with regard to the various kinds of gases depend on the temperature of the sensitive film 105. Therefore, the kinds of gases included in an atmosphere can be identified and concentrations thereof can be identified by knowing the temperature dependency of the sensitivity of the sensitive film 105 for respective gases as shown by FIG. 32 and by comparing the known sensitivities with values of the change in the resistance of the sensitive film 105 at a plurality of temperatures in the atmosphere being tested. The gas concentration of a single gas can also be detected.

Before detecting the change in the resistance of the sensitive film 105, the temperature of the sensitive film 105 is temporarily set to a predetermined temperature. According to the gas sensor of FIG. 30, by changing the temperature of the heater 103, the temperature of the sensitive film 105 can similarly be changed, and the temperature of the sensitive film 105 is thus controlled by controlling temperature of the heater 103.

FIG. 33 shows a specific method of temperature control of the heater 103. As shown by FIG. 33, the temperature of the heater 103 is varied to several temperatures (hereinafter, heater detection temperatures) H1 through H6, and the temperature is temporarily set to a reference heater temperature H0.

Thus, between the times when the sensitive film 105 is changed to the respective heater detection temperatures H1 through H6 (hereinafter, referred to as detection temperatures H1 through H6), the temperature temporarily changes to the reference heater temperature H0 (sometimes referred to as the reference sensitive film temperature). The changes in the resistance of the sensitive film 105 are measured when the temperature reaches the respective detection temperatures.

That is, before detecting the changes in the resistance of the sensitive film 105 at the plurality of respective detection temperatures, the temperature of the sensitive film 105 is temporarily changed to the reference sensitive film temperature, and when the temperature of the sensitive film 105 is changed from a given detection temperature to the next detection temperature, the temperature is temporarily changed to the reference sensitive film temperature as indicated in FIG. 33.

The reference heater temperature H0 is higher than all of the respective heater detection temperatures H1 through H6. Specifically, the reference sensitive film temperature is equal to or higher than a temperature at which a gas adsorbed to the sensitive film 105 is desorbed from the sensitive film 105, that is, equal to or higher than a temperature at which no change in the resistance of the sensitive film 105 is caused by adsorbing the gas. It is preferred that the reference sensitive film temperature is equal to or higher than a temperature at which moisture adsorbed to the sensitive film 105 is desorbed from the sensitive film 105.

However, the temperature of the heater 103 is maintained lower than an ignition temperature, which depends on the environment in which the gas sensor is used. This is to eliminate the possibility of causing a fire. It is preferred to control the heater temperature to the lowest ignition temperature or lower assuming that the concentration of a flammable gas is elevated to an ignition concentration, even when the concentration of the flammable gas of the atmosphere is equal to or lower than the ignition concentration. By controlling the temperature of the heater 103 in this way, it is not necessary to provide a combustion-proof construction for the gas sensor. A reference sensitive film temperature, for example, about 500° C. or lower is preferred. The detection temperatures of the sensitive film are, for example, in the range of about 200° C. to 450° C.

The sensitive film 3 is held to the reference sensitive film temperature for a predetermined period of time by holding the heater 103 to the reference heater temperature H0 for the predetermined period of time. The predetermined time period is a time period sufficient for the resistance of the sensitive film 105 to stabilize. After the resistance is stabilized, the temperature of the sensitive film 105 is changed to the detection temperature. At the reference sensitive film temperature, the time period for the resistance of the sensitive film 105 to stabilize is, for example, about 10 sec.

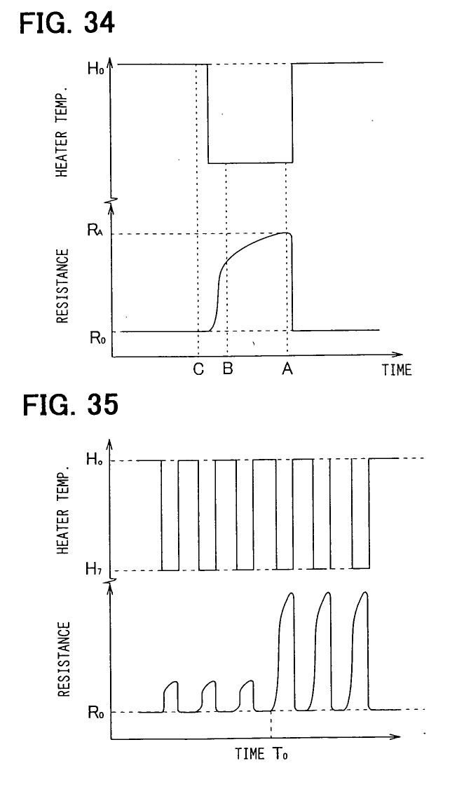

The change in the resistance of the sensitive film 105 is measured after holding the temperature of the sensitive film 105 at the detection temperature for the predetermined time period by changing the temperature of the heater 103 to the respective heater detection temperatures H1 through H6 and thereafter by holding the heater 103 at the respective temperatures for the predetermined time period. Specifically, the change in the resistance of the sensitive film 105 is measured after the resistance of the sensitive film 105 has stabilized. That is, the resistance of the sensitive film 105 is measured at time A in FIG. 34. The time period necessary for the resistance of the sensitive film 105 to stabilize is, for example, about 10 sec.

Temperature control of the heater 103 is performed by the heater temperature control circuit 201, and detection of the change in the resistance of the sensitive film 105 is performed by the sensitive film change analyzing circuit 202. As shown by FIG. 34, a value RA of the change in the resistance at the respective heater detection temperature is measured. That is, the change in resistance is measured with respect to R0. Thereafter, from a relationship between the sensitive film temperature and the resistance change, which is previously known, the kind of a gas and the concentration of the gas are identified (or at least one of these is identified).

Thus, by temporarily setting the temperature of the sensitive film 105 to the reference sensitive film temperature, the sensitive film 105 can be returned to a predetermined reference state. Therefore, the problem of the history of the sensitive film 105 affecting the measurement when the temperature of the sensitive film is changed to a plurality of the detection temperatures, that is, the influence of gas adsorbed to the sensitive film 105 when the temperature of the sensitive film 105 is directly changed to successive detection temperatures, is avoided. That is, the effect of the gas adsorbing history of the film 105 on the change in the resistance of the sensitive film 105 is avoided. Therefore, the kind of gas and concentration of gas can be identified accurately.

The temperature of the sensitive film 105 is set to the reference sensitive film temperature every time before the temperature is set to the respective detection temperature. Therefore, the change in the resistance can always be detected as a change from the same reference resistance R0 and, as a result, the detection is more accurate.

Since the reference sensitive film temperature is higher than all of the respective detection temperatures, desorption of gases or moisture present at the surface of the sensitive film 105 is improved and the sensitive film 105 is brought into a predetermined state in a short period of time. Desorption of gases or the like adsorbed in the surface of the sensitive film 105 is rapid because of the high temperature. Therefore, gases can be identified quickly.

When gas or moisture is adsorbed in the sensitive film 105, the sensitivity of the sensitive film 105 deteriorates. Therefore, the reference sensitive film temperature is equal to or higher than a temperature at which gases or moisture that have been adsorbed in the sensitive film 105 are desorbed from the sensitive film 105. Thus, deterioration in the sensitivity of the sensitive film 105 is inhibited by returning to an initial state at which gas or moisture is not adsorbed in the sensitive film 105.

Since the initial state is a state in which the resistance of the sensitive film 105 is not affected by adsorption, it is not necessary to measure the resistance when the temperature of the sensitive film 105 is set to the reference sensitive film temperature and only the change in the resistance at the detection temperature may be measured.

Since the sensitive film 105 is held at the reference sensitive film temperature for the predetermined time period, gases or moisture will be desorbed from the sensitive film 105. When moisture or gases are completely desorbed from the surface of the sensitive film 105, the resistance of the sensitive film 105 is stable. Therefore, the temperature of the sensitive film 105 can be set to the detection temperature after confirming that moisture or gases have been desorbed from the surface of the sensitive film 105 by setting the temperature of the sensitive film 105 to the detection temperature after the resistance of the sensitive film 105 has been stabilized by setting the temperature of the sensitive film 105 to the reference sensitive film temperature. Therefore, the change in the resistance can be measured with higher accuracy.

The change in the physical value of the sensitive film 105 is detected after the predetermined time period has elapsed when the temperature of the sensitive film 105 is set to the detection temperature, specifically, after the resistance of the sensitive film 105 has been stabilized. Therefore, the resistance can be measured with high accuracy, as when the sensitive film 105 is set to the reference sensitive film temperature.

A cycle of changing the temperature of the heater 103 (temperature of the sensitive film 105) shown in FIG. 33, can determine the kind and the number of gases in the environment to be tested, and the cycle of temperature change shown in FIG. 33 may be repeated.

### Fifteenth Embodiment

According to the fifteenth embodiment, the temperature of the sensitive film 105 is repeatedly set to a constant detection temperature. Mainly, parts that differ from the fourteenth embodiment are described.

For example, when there is only one kind of a gas in the environment being tested, the gas sensor is used to measure only the concentration of the gas. In this case, the concentration of the gas can be identified by measuring the resistance of the film 105 while repeatedly setting the temperature of the sensitive film 105 to the same temperature as indicated in FIG. 35. As shown by FIG. 35, the temperature of the heater 103 is set to the reference heater temperature H0 between instances of changing the temperature of the heater 103 to a fixed temperature (heater detection temperature) H7. At the detection temperature H7, the resistance of the sensitive film 105 is measured.

Since the variation in the resistance of the sensitive film 105 changes from time T0, it is known that the gas concentration of the atmosphere changes from the time T0. Since the relationship between resistance and gas concentration is known, the gas concentration before and after the time T0 can be identified.

For the reasons given with respect to the fourteenth embodiment, the gas can be identified quickly and accurately.

Further, the time period during which the temperature is held steady at the reference sensitive film temperature and at the detection temperature and a time point for measuring the resistance at the detection temperature are similar to that in the fourteenth embodiment.

### Sixteenth Embodiment

In the sixteenth embodiment the point in time when the resistance of the sensitive film 105 is measured is different from that in the fourteenth and fifteenth embodiments. It is known that the rate of the change in the resistance varies depending on the concentration of the gas. Specifically, the time period during which the resistance of the sensitive film 105 changes is relatively short when the concentration of the gas is relatively great.

Therefore, when the relationship between the rate of change in the resistance of the sensitive film 105 and the concentration of the gas is known, the concentration of the gas can be identified before the resistance stabilizes.

However, the change in the rate of change of the resistance differs between a case in which the concentration of the gas is changed from, for example, 5% to 20% and a case in which the concentration is changed from 10% to 20%. Hence, by setting the temperature of the sensitive film 105 temporarily to the reference sensitive film temperature before the temperature of the sensitive film 105 is set to the detection temperature, as in the fourteenth and fifteenth embodiments, the change in the rate of the change in the resistance is accurately detected. Therefore, when the relationship between the change in the rate of the change in the resistance and the concentration of the gas is known, the concentration of the gas can be identified.

Next, an explanation will be given of the specific time point at which the resistance is measured. The slope of the change in the resistance can be measured at time B in FIG. 34, for example, which is a time point before the change in the resistance of the sensitive film 105 is stabilized. The time B may be a certain time from when a switch was made to change the temperature of the sensitive film 105 to the detection temperature. The change in the resistance at the same time is previously measured to prepare a reference data base and stored for access by the sensitive film change analyzing circuit 202. Thus, the concentration of the gas is identified by comparing the slope of the change in the resistance in the data base with that measured at the time B.

In this embodiment, it is not necessary to wait until the resistance of the sensitive film 105 stabilizes. Thus, the gas can be identified quickly. Specifically, after the temperature of the sensitive film 105 is set to the reference sensitive film temperature, the temperature of the sensitive film 105 is set to the detection temperature. After measuring the slope of the change in the resistance, before the resistance of the sensitive film 105 stabilizes, the temperature of the sensitive film 105 is immediately changed to the reference sensitive film temperature. Then, the temperature of the sensitive film 105 is again set to the detection temperature to repeat the detection. Therefore, the gas can be identified more quickly, and the advantages of the fourteenth and the fifteenth embodiments are achieved.

### Seventeenth Embodiment

FIGS. 37 and 38 show a thin-film type sensor (hereinafter, simply referred to as a gas sensor) 300 according to the seventeenth embodiment. As shown by FIG. 37 and FIG. 38, a sensitive film 302, a physical value of which is changed by a reaction with a gas, is located on a substrate 301. The substrate 301 is, for example, an amorphous alumina substrate, and the depth of recesses and the height of projections from the surface of the substrate 301 are equal to or less than ⅕ of the thickness of the sensitive film 302. The sensitive film 302 is tin oxide, and the thickness of the sensitive film 302 is several nanometers.

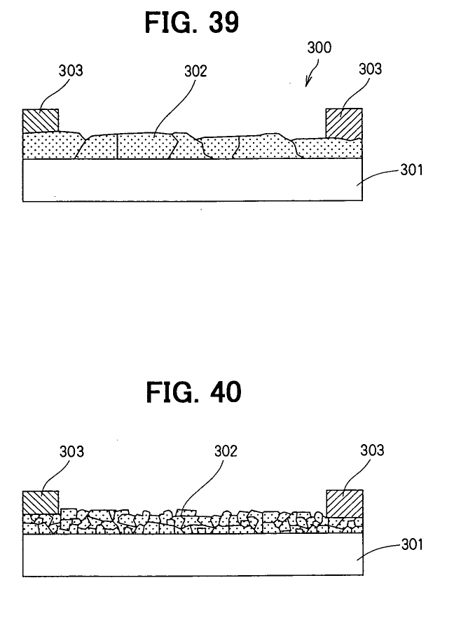

As shown by FIG. 39, the average crystal grain diameter (hereinafter, simply referred to as average grain diameter) of the sensitive film 302 is equal to or larger than the film thickness of the sensitive film 302. The average grain diameter D is provided by an intercepting method for identifying grain diameter, and when the average film thickness of the sensitive film 302 is designated by T, the average grain diameter is equal to or larger than the film thickness, or D≧T.

A pair of electrodes 303, for detecting the change in the physical value of the sensitive film 302, is formed on the sensitive film 302. Each of the electrodes 303 is formed in a comb-like shape as shown. Ends of the electrodes 303 extend toward the periphery of the sensitive film 302 and are connected to electrode pads 303a. The electrodes 303 are, for example, platinum.

A heater 304, or heater layer, which is for heating the sensitive film 302, surrounds the sensitive film 302 and the detection electrodes 303 on the surface of the substrate 301. The heater 304 has a frame shape, and heater pads 304a extend from the heater 304 as shown. The heater 304 may be made of, for example, platinum.

Although not illustrated, the heater pads 304a are connected to a sensitive film temperature control circuit for adjusting the temperature of the sensitive film 302 by adjusting the heat generation of the heater 304, and the electrode pads 303a are connected to a sensitive film change analyzing circuit for detecting a change in the physical value of the sensitive film 302.

Next, a method of fabricating the gas sensor 300 will be described. First, the substrate 301 is prepared and a substrate processing step is performed for reducing the sizes of recesses and projections of the surface. Generally, in a commercially available alumina substrate, since recesses and projections in the surface are as large as about several 10 through 100 nm and the surface is contaminated by a carbide or the like, the coalescence of grains in initial formation necessary for forming the sensitive film 302 having a large grain diameter is hindered. Hence, by reducing the recesses and projections of the surface of the substrate 301, the sensitive film 302 can be formed with a large grain diameter.

Specifically, the surface of the substrate 301 is mechanically polished and repeated acidic cleaning, alkaline cleaning or the like are performed in the substrate processing step. By reducing the recesses and projections of the surface of the substrate 301 to have dimensions from the surface of the substrate that are equal to or less than ⅕ of the film thickness of the sensitive film 302, the average grain diameter of the sensitive film 302 can be equal to or larger than the film thickness of the sensitive film 302.

Next, the sensitive film 302 is formed on the substrate 301. Specifically, the sensitive film 302 is deposited over a face of the substrate 301 by an atomic layer growing method, which includes alternately supplying tin chloride, which is a gas that includes a metal for making the sensitive film 302, and water to the substrate 301. The processing temperature in this step is about 200 to 300° C., and the sensitive film 302 is deposited with a thickness of about several nanometers. Thereafter, a heat treatment of about 500° C. is carried out on the sensitive film 302 in an oxygen atmosphere.

By forming the sensitive film 302 by alternately supplying tin chloride and water, when tin chloride is introduced, one atomic layer of tin can be deposited on the substrate 301, and when water is introduced, one atomic layer of oxygen can be deposited, and the sensitive film 302 can be formed to have a stoichiometric ratio from the initial stage of growth.

That is, the sensitive film 302 can be formed by controlling the composition by depositing the sensitive film 302 by a unitary atomic layer. Therefore, the sensitive film 302 will have the desired crystalline structure without depending on the kind of the substrate 301.

Therefore, a fine crystal formation of the sensitive film 302 like that in FIG. 40 is prevented The fine crystal formation of FIG. 40 is caused by depositing the sensitive film 302 by a method, such as sputtering or the like, in which the composition ratio cannot be accurately controlled. As a result, the sensitive film 302 has a large grain diameter, and the average grain diameter of the sensitive film 302 is equal to or larger than the film thickness of the sensitive film 302.

Next, the sensitive film 302, which is formed over one face of the substrate 301, is patterned into a desired shape by selectively carrying out dry etching (reactive etching) by using an etching gas mixed with argon and chlorine gas, in a well-known lithography technique.

Next, a platinum film, for forming the heater 304 and the electrodes 303, is formed in a thickness of about 250 nm by a vapor deposition process and is patterned by using a lithography technique. That is, the platinum film is patterned into the shapes of the heater 304, the electrodes 303 and the respective pads 303a and 304a by a dry etching process using argon gas.

At this point, a titanium film (not illustrated) may be deposited in a thickness of about 5 nm below the platinum film to promote adherence between the substrate 301 and the heater 304 and between the sensitive film 302 and the electrodes 303. Thus, exfoliation of the heater 304 and the electrode 303 when the heating cycles occur, can be significantly reduced.

Thereafter, the heater pad 304a is connected to the sensitive film temperature control circuit by a bonding wire or the like, and the electrode pad 303a is connected to the sensitive film change analyzing circuit.

In the gas sensor 300, the temperature of the sensitive film 302 is changed to a desired temperature by the sensitive film temperature control circuit, and the physical value of the sensitive film 302, when the temperature of the sensitive film 302 reaches the desired temperature or when a certain desired time is reached, is inputted as a signal and analyzed by the sensitive film change analyzing circuit.

Changes in the physical value of the sensitive film 302 depend on the temperature of the sensitive film 302, and the dependency of the change in the physical value on the temperature differs according to the gas being detected. Therefore, by measuring the change in the physical value of the sensitive film 302 at various temperatures, the concentration of the gas or the identity of the gas can be specified. The detected physical value can be the resistivity of the sensitive film 302, the change in the dielectric constant, the thermal conductivity, or the like, which are varied by adsorption and desorption of a gas.

When variation of the change in the resistivity is measured by actually changing the mean grain diameter of the sensitive film 302, the larger the mean grain diameter, the faster the response speed, and the response is significantly improved when the average grain diameter becomes larger than the film thickness.

This seems to be because the change in a characteristic of the sensitive film 302 by gas adsorbed to and desorbed from the surface becomes stronger when the grain boundaries are reduced. By forming the sensitive film 202 with a large grain diameter, the problems of grain growth progressing in heating and deterioration of stability with age are reduced.

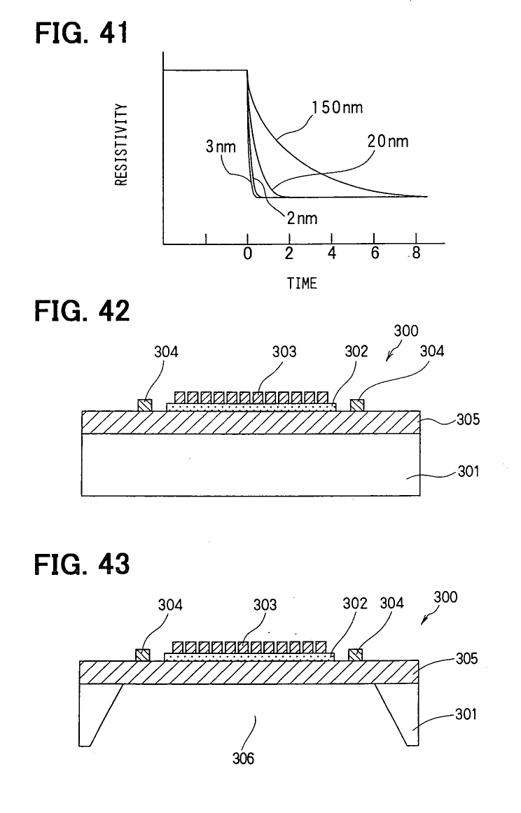

An investigation was carried out of the response of the gas sensor 300 when the film thickness of the sensitive film 302 varies. Specifically, the change in time of the change in the resistivity was measured when hydrogen having a concentration of 1% was used as the gas to be detected. The result is shown in FIG. 41. The results show that the smaller the film thickness of the sensitive film 302 was, the higher the response was. Further, the detection sensitivity (change in resistivity) was also increased.

Particularly, when the film thickness was equal to or smaller than 12 nm, that is, equal to or smaller than the thickness of a depletion layer produced by adsorption of the gas to be detected in the sensitive film 302, the response and the sensitivity become extremely high. However, when the film thickness is equal to or smaller than 3 nm, the sensitive film is liable to be damaged by thermal stress, due to a difference of its thermal expansion coefficient from that of the matrix substrate 301. Therefore, it is preferred that the film thickness of the sensitive film 302 be in a range of 3 nm to 12 nm.

Thus, the crystal grain boundary is reduced by enlarging the average grain diameter of the sensitive film 302, which is not dependent on the kind of the substrate 301 being used. Therefore, the gas sensor will be very responsive.

Therefore, a highly responsive gas sensor 300 is produced without using a special substrate, such as an insulating substrate of a single crystal, and the gas sensor 300 is relatively inexpensive, since single insulating substrates are generally expensive.

### Eighteenth Embodiment

With reference to FIG. 42, the eighteenth embodiment employs a silicon substrate of a single crystal. The following description will mainly cover parts that differ from the seventeenth embodiment, and parts of FIG. 42 that are the same as those in FIG. 38 have the same reference numbers.

As shown by FIG. 42, an insulating film 305 is formed on the substrate 301. The insulating film 305 is a silicon oxide film or a silicon nitride film, which are known in the field of semiconductor fabrication. The sensitive film 302 and the heater 304 are formed above the substrate 301 and above the insulating film 305. The substrate 301 is electrically insulated from the sensitive film 302.

When an oxide film, particularly a thermal oxide film, is used as the insulating film 305, adherence to the substrate 301 is ensured and reliability is improved. The sensitive film 302 is formed on the insulating film 305 in a manner similar to that of the seventeenth embodiment.

Thus, even when the silicon substrate 301, which is easy to obtain at low cost, is used, the average grain diameter of the sensitive film 302 can be enlarged and the gas sensor 300 will be highly responsive.

The flatness of the surface of the silicon substrate is inherently high, and therefore, the substrate processing step of the seventeenth embodiment is not necessary.

### Nineteenth Embodiment

FIG. 43 shows a gas sensor 300 in which a hollow cavity 306 is formed by removing a portion of the substrate 301 that corresponds to the sensitive film 302. The substrate 301 may be made of silicon (100), and the insulating film 305 is formed above the substrate 301. The insulating film 305 is formed by laminating a silicon nitride film, a silicon oxide film, a silicon nitride film and a silicon oxide film in this order. The sensitive film 302 and the heater 304 are formed on the insulating film 305. Further, by forming the cavity portion 306 in the substrate 301, the insulating film 305 spans an opening of the hollow cavity 306 that is on the side of the sensitive film 302.

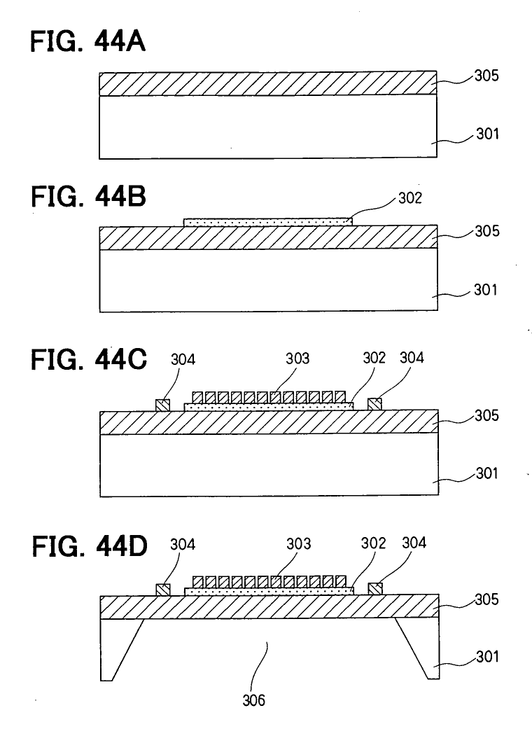

Next, a method of fabricating the gas sensor 300 will be described with reference to FIGS. 44A, 44B, 44C and 44D.

Step of FIG. 44A

The insulating film 305 is formed on the substrate 301.

Specifically, the silicon nitride film is formed with a thickness of 120 nm by an LP-CVD process. Then, the silicon oxide film is deposited with a thickness of 1 μm by a plasma CVD process. Thereafter, after forming another silicon nitride film with a thickness of 130 nm by LP-CVD, the silicon nitride film is thermally oxidized to change a very thin layer at the surface of the silicon nitride film into a silicon oxide film.

Step of FIG. 44B

A sensitive film is formed in the manner of the seventeenth embodiment.

Step of FIG. 44C

The heater 304 and the electrode 303 are formed in the manner of the seventeenth embodiment.

Step of FIG. 44D

An oxide film is formed by a plasma CVD process on a face of the substrate 301 that is opposite to the sensitive film 302 to form a mask (not illustrated). Then, the hollow cavity 306 is formed by etching the substrate 301 by a TMAH solution using the mask.

Thus, the insulating film 305 spans an opening of the hollow cavity 306, and since the insulating film 305 is made by laminating the silicon nitride films and the silicon oxide films, cambering at the spanned location is reduced. Since tensile stress is applied to the insulating film 305, the insulating film 305 and the sensitive film 302 and the like formed on the insulating film 305 are not damaged by buckling.

By providing the hollow cavity 306, heat transfer of heat generated from the heater 304 layer through the substrate 301 is limited. Therefore, the power necessary for heating the sensitive film 302 can be significantly reduced, and the gas sensor 300 is more efficient and more responsive.

When the sensitive film 302 is heated intermittently in detecting a gas, by providing the hollow cavity, the intermittent operation can be made very fast. Since the sensitive film 302 is formed on the silicon oxide film, exfoliation of the sensitive film 302 and the insulating film 305 is greatly reduced.

When the hollow cavity 306 is formed, instead of the TMAH solution, a KOH solution or the like constituting a strong alkaline solution can be used.

The hollow cavity 306 need not be formed by etching the substrate 301 until the insulating film 305 is exposed. A similar effect can be achieved by constructing a thin-walled structure in which the thickness of the substrate 301 is reduced more than another portion of the substrate 301 at the location of the substrate 301 that corresponds to the sensitive film 302. In this case, it is preferred that the thickness of the thin-walled structure portion is, for example, about several micrometers.

### Twentieth Embodiment

In this embodiment, a singe-crystal silicon substrate is employed as the substrate 301, and this embodiment differs from those of the eighteenth and nineteenth embodiments in the nature of the insulating film 305. The cross section of the gas sensor 300 according to the twentieth embodiment is similar to that in FIG. 42 or FIG. 43 and thus will not be described.

In the eighteenth and nineteenth embodiments, an amorphous layer such as the silicon oxide film or a silicon insulating film is employed as the insulating film 305 on the substrate 301. However, according to this embodiment, a single crystal, which is made to grow heteroepitaxially on the substrate 301, is used. Specifically, an Al₂O₃ film can be used as the insulating film 305. The sensitive film 302 is formed on the insulating film 305.

To form such an insulating layer 305, first, a γ-Al₂O₃ layer is formed with a thickness of 100 nm at about 900° C. by a CVD process using TMA and N₂O gas. The lattice mismatch between the γ-Al₂O₃ layer and the silicon (Si) substrate is as small as about 2% and the γ-Al₂O₃ layer can be made to grow epitaxially such that a (100) face of the silicon substrate and a (100) face of the γ-Al₂O₃ layer are parallel.

By forming tin oxide on the γ-Al₂O₃ film by an atomic layer growing method, the grain diameter of the sensitive film 302 can be made larger than that where the sensitive film 302 is formed on the silicon oxide film as in the eighteenth and nineteenth embodiments.

It seems that, on an amorphous layer of the silicon oxide film or the like, as described above, the tin oxide film has a high orientation performance and, by forming the sensitive film 302 on the γ-Al₂O₃ film, a film near to an epitaxially-formed layer can be formed, and the grain diameter can be increased. By making the grain diameter of the sensitive film 302 large, the grain boundary can be reduced, which improves the responsiveness of the gas sensor 300.

Other than the γ-Al₂O₃, even when a CaF₂ film or a CeO₂ film is formed by an atomic layer growing method to create the insulating film 305, the lattice constants of the films are near to that of the silicon substrate. Therefore, a film having few defects is formed. The insulating film 305 can be formed with high flatness by employing the atomic layer growing method. As a result, control of composition of the sensitive film 302 can be carried out with extremely high accuracy and therefore, the gas sensor 300 will be very responsive even when the CaF₂ film or the CeO₂ film is used as an insulating film 305.

The whole substrate is not made with an expensive insulating single crystal substrate, as in the technology described in the above-described prior art method, but the single crystal insulating film 305 is formed above the inexpensive silicon substrate. Therefore, a highly responsive gas sensor is provided at low cost.

Other insulating substances (other than the y-Al₂O₃ film, the CaF₂ film, or the CeO₂ film) that can be made to grow epitaxially can be used as the insulating film 305.

### Twenty-First Embodiment

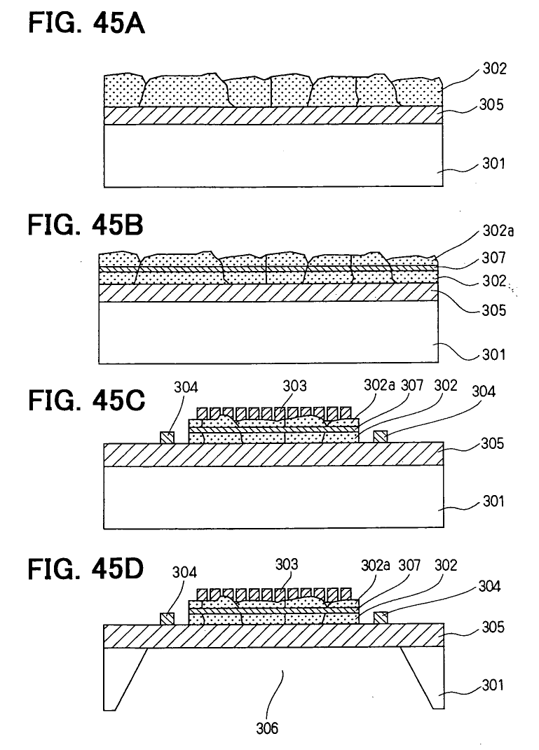

According to the twenty-first embodiment, an insulating layer is inserted into the sensitive film 302 for improving the responsiveness. This embodiment is described with reference to FIGS. 45A, 45B, 45C and 45D, which show a method of fabricating the gas sensor 300. Mainly, parts that differ from the nineteenth embodiment will be described, and parts of FIGS. 45A, 45B, 45C and 45D that are the same as corresponding parts of FIGS. 44A, 44B, 44C and 44D have the same reference numbers.

As shown by FIG. 45D, which illustrates a finished gas sensor 300 according to this embodiment, an ion-implanted layer (insulating layer) 307, which has an electric conductivity that is less than that of the sensitive film 302, is formed in a mid-section of the crystal grains in the sensitive film 302.

Next, a method of fabricating the gas sensor 300 of FIG. 45D will be described.

Step of FIG. 45A

Like the nineteenth embodiment, the insulating layer 305 is formed by the silicon nitride films and the silicon oxide films on the substrate 301 of silicon (100). Then, the sensitive film 302, which includes tin oxide, is formed by the atomic layer growing method as in the above-described sensitive film forming step. However, the sensitive film 302 has a thickness of 0.8 μm.

The average grain diameter of the sensitive film 302 is about 1 μm, which is generally larger than the film thickness of the sensitive film 302.

Step of FIG. 45B

Next, ions are implanted in the sensitive film 302 to form the ion-implanted layer 307. Specifically, a tin-enriched ion-implanted layer 307, which is near to amorphous and substantially parallel with the substrate 301, is formed. The ion-implanted layer 307 is formed using tin ions in the sensitive film 302 at a depth of about 0.2 μm from the surface of the sensitive film 302 and is located in a middle section as shown. Then, heat treatment is performed at about 500° C., again, in an oxygen atmosphere, to reduce defects.

Step of FIG. 45C

Next, a resist is formed on the sensitive film 302. The resist is patterned by using photolithography, and thereafter, the sensitive film 302 is patterned in a desired shape by etching through the resist to finish the sensitive film 302. Therefore, the ion implanting step is carried out in the sensitive film forming step.

Then, the heater 304 and the electrode 303 are formed in the manner of the nineteenth embodiment.

Step of FIG. 45D

Subsequently, the hollow cavity 306 is formed in the manner of the nineteenth embodiment. Then, the gas sensor 300 is finished.

By forming the ion-implanted layer 307 in the sensitive film 302, only a sensitive film upper layer portion 302a (a portion having a thickness of 0.2 μm) substantially functions as the sensitive film 302, and adsorption and desorption of the gas to be detected is carried out at the sensitive film upper layer portion 302a.

Since the ion-implanted layer 307 is formed at the middle of the crystal grains, at the sensitive film upper layer portions 302a, the crystal grain diameter is much greater than the film thickness. Therefore, the response is further improved by further reducing the number of crystal grains.

The ion-implanted layer 307 can be formed at a shallower level of the sensitive film 302 by lowering the acceleration voltage in the ion plantation or by implanting ions in a state in which the surface of the sensitive film 302 is covered by a silicon oxide film or the like. By forming the ion-implanted layer 307 and further decreasing the film thickness of the part functioning as the sensitive film 302, higher sensor responsiveness is achieved. Furthermore, the detection sensitivity can be increased by increasing the change in the resistivity by adsorption and desorption of the gas to be detected.

Particularly, when the film thickness of the part functioning as the sensitive film 302 is made equal to or smaller than 10 nm, that is, equal to or smaller than the thickness of the depletion layer produced by permitting a gas to be adsorbed by the sensitive film 302, the gas sensor 300 will be very sensitive and responsive.

Although tin atoms are employed for forming the ion-implanted layer 307, any atom, such as silicon, aluminum or the like, that is capable of making the ion-implanted layer 307 act as insulation may be used.

Even when the average crystal grain diameter of the sensitive film, is small, the responsiveness of the gas sensor can be improved by adjusting the level of the ion-implanted layer 307 in the sensitive film 302 such that the average grain diameter of the sensitive film upper layer portion 302a is equal to or larger than the film thickness of the sensitive film upper layer portion 302a.

### Twenty-Second Embodiment

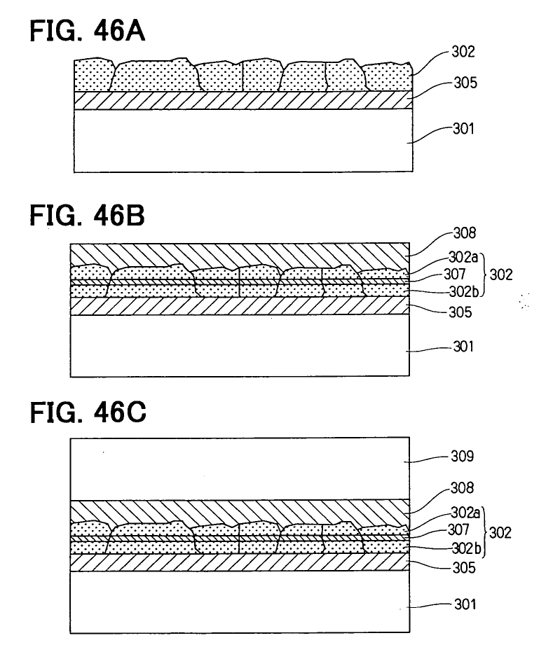

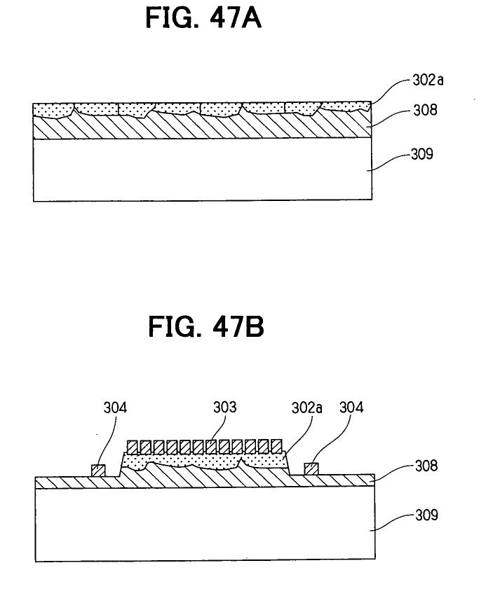

The twenty-second embodiment is an alternative method of fabricating the sensitive film 302 such that the average grain diameter of the sensitive film 302 is equal to or larger than the film thickness of the sensitive film 302. FIGS. 46A, 46B and 46C show steps of the method of fabricating the sensitive film according to the twenty-second embodiment, and FIGS. 47A and 47B show steps subsequent to FIG. 46C.

Step of FIG. 46A

As in the twenty-first embodiment, the insulating film 305, which includes the silicon nitride films and the silicon oxide films, is formed on the substrate 301 of silicon (100), and the sensitive film 302 is formed on the insulating film 305. The sensitive film 302 has a thickness of 0.8 μm. The average grain diameter of the sensitive film 302 is about 1 μm as in the twenty-first embodiment.

Step of FIG. 46B

Next, ion implantation is performed for forming the ion-implanted layer 307 substantially parallel to the substrate 301 at the middle of the sensitive film 302. Hydrogen ions are used as the ions, and the ion-implanted layer 307 is formed in the sensitive film 302 at about 0.15 μm from the surface by the ion implanting method.

Then, a silicon oxide film 308 is deposited with a thickness of about 5 μm on the sensitive film 302 at about 300° C. by a plasma CVD process. To flatten recesses and projections on the surface of the silicon oxide film 308, polishing or the like is carried out to make the film thickness of the silicon oxide film 308 about 2 μm.

Step of FIG. 46C

Next, a surface of a silicon substrate (hereinafter, referred to as the other substrate) 309, which is prepared separately, is thermally oxidized to form an oxide film, and the oxide film and the surface of the silicon oxide film 308, which was flattened in the Step of FIG. 46B, are pasted together by removing water between the two oxide films at about 300° C.

Step of FIG. 47A

Next, the ion-implanted layer 307 is heat treated while the substrates 301 and 309 are pasted together. As a result, the ion-implanted layer 307 becomes brittle and the sensitive film 302 is divided at the ion-implanted layer 307.

Step of FIG. 47B

By using the sensitive film upper layer portion 302a formed at the other substrate 309, a resist is patterned on the sensitive film upper layer portion 302a by photolithography, and the sensitive film upper layer portion 302a is patterned in a desired shape.

The sensitive film 302 is formed on the insulating film 305 in the step shown in FIG. 46A, and the sensitive film upper portion 302a is patterned in a desired shape at the step shown in FIG. 47B. That is, in the sensitive film forming step, the ion implanting step and the step of dividing the sensitive film are performed.

Thereafter, by forming the heater 304 and the electrode 303 in the manner of the twenty-first embodiment, the gas sensor 300 is finished.

By fabricating the gas sensor 300 by such a method, which is similar to the method of the twenty-first embodiment, the sensitive film upper layer portion 302a (which has a thickness of 0.15 μm in this embodiment) can be used as the sensitive film. Therefore, the advantages of the twenty-first embodiment are similarly achieved.

The ion implanting step can be carried out after depositing the silicon oxide film 308 on the surface of the sensitive film 302 and after flattening the silicon oxide film 308. Thus, ions can be implanted from the flattened face, and therefore, the ion-implanted layer 307 can be formed flatly. As a result, the dividing face of the sensitive film 302 is flat, and accordingly, the electrode 303 can be formed on the flat face and the connection reliability of the electrode 303 can be improved.

In dividing the sensitive film 302, the substrates 301 and 309 may be pasted together by using polycrystalline silicon or AuSi eutectic.

By laminating the films formed at the other substrate 309 in the order of a silicon oxide film, a silicon nitride film and a silicon oxide film to provide tensile stress to the films, the hollow cavity 306 can be formed in the manner of the nineteenth embodiment.

The ion-implanted layer 307 may be formed at a shallower level of the sensitive film 302 as described in the twenty-first embodiment.

Although use of the sensitive film upper layer portion 302a by dividing the sensitive film 302 has been shown, a sensitive film lower layer portion 302a located below the ion-implanted layer 307 in the sensitive film 302 may be used.

Even when the average crystal grain diameter of the sensitive film formed in the sensitive film forming step is small, a responsive gas sensor can be formed by adjusting the position of the ion-implanted layer 307 in the sensitive film 302 such that the average grain diameter is equal to or larger than the film thickness of at least one of the sensitive film upper layer portions 302a or the sensitive film lower layer portion 302b in the ion implanting step.

### Twenty-Third Embodiment

When the gas to be detected is hydrogen gas, to reduce the influence of other gasses, a filter layer, such as an SiO₂ film, an Al₂O₃ film or the like for permitting hydrogen gas to selectively pass, promotes selectivity. However, when such a filter layer is formed, there is a concern that the sensitive film 302 cannot be fully covered due to the surface state of the sensitive film 302 when a normal film forming method such as sputtering or the like is used. Therefore, it is necessary to increase the thickness of the filter layer to several 100 nm to ensure selectivity by fully covering the surface of the sensitive film 302. However, when the thickness of the filter layer is relatively great, there is a delay before gas is detected, due to the additional time for the gas to reach the sensitive film 302. Thus, the filter lowers responsiveness.

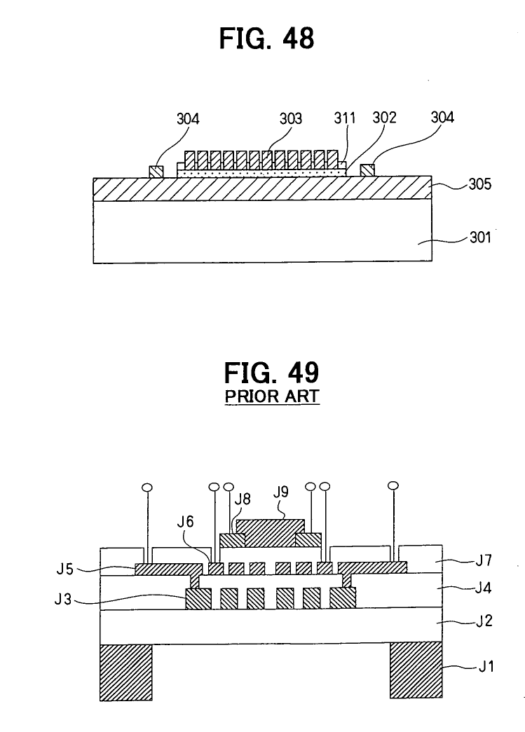

Hence, as shown by FIG. 48, an Al₂O₃ layer is deposited as the filter layer 311 on the surface of the sensitive film 302 by the atomic layer growing method. Thus, since the atomic layer growing method forms a dense filter layer 311, even when the filter layer 311 is thin, the filter layer 311 can fully cover the surface of the sensitive film 302, and high selectivity can be ensured without deteriorating response. To prevent the response from deteriorating while fully covering the surface of the response film 302, the film thickness of the filter layer 311 preferably falls in a range of about 10 nm to 50 nm.

The filter layer 311 may be formed by the atomic layer growing method after the sensitive film is formed. Thereafter, the electrode 303 and the sensitive film 302 are electrically connected by forming contact-holes at the filter layer 311.

Although FIG. 48 shows that the filter layer 311 is formed in the manner of the seventeenth embodiment, a similar effect can be achieved by forming the filter layer 311 in the manner of the seventeenth through twenty-second embodiments.

### Other Embodiments

Although the fourth through the thirteenth embodiments have been described separately, features of the fourth through the thirteenth embodiments can be used in the methods of the first through the third embodiments.

Although electric resistance has been described as the physical value of the electric film 5, dielectric constant, electrostatic capacitance, weight or the like may be detected.

For etching the hollow cavity 8, methods other than anisotropic etching by the TMAH solution can be used as long as the hollow cavity 8 can be formed. Particularly, when corner portions of the heater electrode 3 and the sensitive film 5 are chamfered or rounded to form shape the hollow cavity 8 in the manner of the seventh embodiment and the eighth embodiment, anisotropic etching using face orientation may not be performed.

According to the first embodiment, the surface of the flattened insulating layer 9 is flattened. However, a surface of the electrically insulating layer 4 may be flattened and the detection electrodes 6a, 6b and the flattened insulating layer 9 may be formed on the electrically insulating layer 4.

The flattening step according to the first and the second embodiments need not be finished by only polishing or the like, but chemical flattening after polishing can be employed. Such chemical flattening may be carried out without polishing. Further, it is preferred to remove natural oxide film or nitride film on the surfaces of the detection electrodes 6a, 6b, and the films may be removed by using by, for example, hydrogen fluoride or phosphoric acid.

According to the first embodiment, a filter is not employed, however, a filter 12 may be formed in the manner of the second and the third embodiments. Although according to the second and the third embodiments, the filter 12 is employed, the filter 12 need not be employed when the sensitive film 5 is selective for a specific gas. To provide the sensitive film 5 with the selectivity, an impurity reacting with the specific gas may be added to the sensitive film 5.

The hollow cavity 8 exposing the support film 2 need not be formed as described above. The hollow cavity 8 may be formed such that a thin wall of the substrate 1 remains at the upper end of the hollow cavity 8. In this case, the remaining thin-wall of the substrate 1 may be used as a substitute for the support film 2, and the step of forming the support film 2 is not necessary.

In the fourteenth embodiment, stabilization of the change in the resistance can be confirmed for respective different detection temperatures by measuring repeatedly the change in the resistance for respective different detection temperatures and comparing changes in the respective resistance values.

The sensitive film 105 may be set to the detection temperature after confirming that the sensitive film 105 has been brought completely into an initial state by measuring the change in the resistance at time C in FIG. 34.

In the fourteenth and fifteenth embodiments, the change in the resistance may be measured not only at time A of FIG. 34 but also at time B. In this case, measurement data at two points of time, A and B, is provided, and the gas can be identified with higher accuracy.

In the fourteenth through sixteenth embodiments, the temperature of the sensitive film 105 is set to the reference sensitive film temperature each time before the temperature is set to the detection temperature, however, the sensitive film 105 need not necessarily be set to the reference sensitive film temperature every time. The sensitive film 105 may be set to the reference sensitive film temperature as necessary. For example, the sensitive film 105 may be set to the reference sensitive film temperature at least once.

In the fourteenth through sixteenth embodiments, the reference sensitive film temperature is higher than all the detection temperatures, however, the reference sensitive film temperature need not necessarily be higher than all the detection temperatures. For example, when using a sensitive film 105 that cannot be set to high temperatures, by setting the reference sensitive film temperature to about the highest detection temperature or a temperature lower than the highest detection temperature and holding the sensitive film 105 to the reference sensitive film temperature for a relatively long time period, gases or moisture may reliably be desorbed from the sensitive film 105.

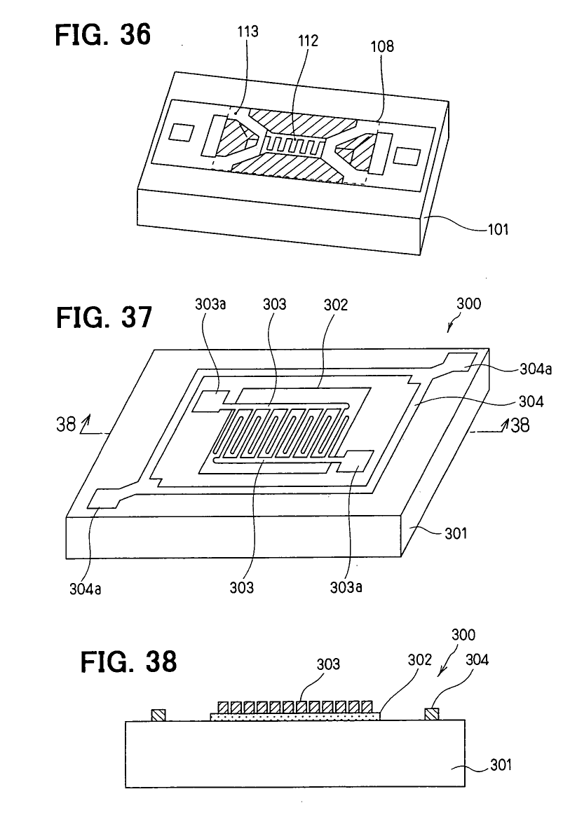

In the fourteenth through sixteenth embodiments, the gas sensor is a membrane type sensor, however, a bridge type gas sensor, as shown in FIG. 36, in which the lower face of the substrate 101 is not opened, can be used. In this embodiment, a hollow cavity 108 opens only at the surface of the substrate 101, and a thin-walled detection portion 112 spans the opening of the hollow cavity 108. The thin-walled detection portion has four connecting portions 113. In FIG. 36, for convenience, the hollow cavity 108 is indicated by hatch lines.

In the fourteenth through sixteenth embodiments, the hollow cavity 108 need not necessarily be provided in the substrate 101. The substrate 101 need not be made of semiconductor substrate. An insulating substrate or the like may also be used.

In the fourteenth through sixteenth embodiments, resistance was suggested as the detected physical value of the sensitive film 105. However, dielectric constant, electrostatic capacitance, weight or the like may be measured instead.

In the seventeenth through twenty-third embodiments, tin oxide is suggested as the material of the sensitive film 302. However, other compounds can be used as long as a physical value of the compound is changed by adsorbing the gas to be detected and as long as the compound can be formed as a film near to an epitaxial film. For example, indium oxide, zinc oxide, tungsten oxide or the like can be used to form the sensitive film 302.

When indium oxide or zinc oxide is used to form the sensitive film 302, the sensitive film 302 can be formed by the atomic layer growing method.

When a highly pure amorphous mullite substrate is used as the substrate 301, since thermal expansion coefficients of the mullite substrate and tin oxide of the sensitive film 302 are very near to each other, exfoliation of the sensitive film 302, which is caused by a difference of thermal expansion between the sensitive film 302 and the substrate 301 when the sensitive film 302 is formed or subjected to a heat treatment, is limited. As a result, the reliability of the gas sensor 300 is improved.

In the seventeenth to twenty-third embodiments, platinum is suggested as the electrode 303 and the heater 304. However, electrically conductive substances other than platinum, such as a lamination of, for example, platinum and titanium or gold or the like, can be employed. Although a titanium film is formed for adhering the electrode 303 and the heater 304 and the matrix film, a material for promoting adhesion, such as chromium or the like, can be used instead. When there is sufficient adhesion between the electrode 303 and the heater 304 and the matrix film, the adhering layer need not be provided.

Instead of detecting the change in the physical value of the sensitive film 302 as an electric signal with the electrode 303, a change in the refractive index of the sensitive film 302 may be detected by light, and any means will do as long as the variation in the physical value of the sensitive film 302 due to diffusion a gas into the sensitive film 302 can be detected.

When the ion-implanted layer 307 is formed in the twenty first and twenty second embodiments, a sensitive film material of a single crystal may be used.

The gas sensor may serve as an odor sensor or a humidity sensor.

1. A gas sensor comprising:
a substrate;
a support film formed on the substrate;
a heater layer formed on the support film;
a first electrical insulation layer facing the heater layer;
a detection electrode supported by the first electrical insulation layer;
a second electrical insulation layer supported by the first electrical insulation layer, wherein the second electrical insulation layer surrounds the detection electrode such that a surface of the detection electrode is exposed, and a surface of the second electrical insulation layer is flat and flush with the surface of the detection electrode; and
a sensitive film formed flatly in contact with the surface of the detection electrode, wherein a physical value of the sensitive film changes when the film reacts to a gas being detected.

2. The gas sensor according to claim 1, wherein the maximum difference between the level of any point on the surface of the detection electrode and that of any point on the surface of the second electrical insulation layer is less than the thickness of the sensitive film.

3. The gas sensor according to claim 1, wherein the gas sensor further includes a hollow cavity formed in the substrate, wherein the hollow cavity is spanned by the support film, and wherein tensile stress equal to or larger than 40 MPa and equal to or smaller than 150 MPa is applied to the support film.

4. The gas sensor according to claim 1 further comprising a filter for permitting a specific gas to reach the sensitive film.

5. The gas sensor according to claim 1, wherein the thickness of the sensitive film is equal to or larger than 3 nm and equal to or smaller than 12 nm.

6. A gas sensor comprising:
a substrate;
a support film formed on the substrate;
a heater layer formed on the support film;
a detection electrode supported by the substrate such that the heater layer and the detection electrode are located on the same surface;
an electrical insulation layer supported by the substrate such that the heater layer is covered by the electrical insulation layer and such that the heater layer is insulated from the detection electrode, wherein a surface of the detection electrode is exposed from the insulation layer, and a surface of the insulation layer is flat and flush with the surface of the detection electrode;
a sensitive film formed flatly in contact with the surface of the detection electrode, wherein a physical value of the sensitive film changes when the film reacts to the gas being detected.

7. The gas sensor according to claim 6, wherein the maximum difference between the level of any point on the surface of the detection electrode and that of any point on the surface of the electrical insulation layer is less than the thickness of the sensitive film.

8. A gas sensor comprising:
a substrate;
a support film formed on the substrate;
an electrical insulation layer supported by the substrate;
a sensitive film formed flatly in contact with the surface of the electrical insulation layer, wherein a physical value of the sensitive film changes when the film reacts to the gas being detected;
a heater layer located above the support film and between the support film and the electrical insulation layer and outside of an imaginary normal projection of the sensitive film; and
a detection electrode formed on the sensitive film for detecting a change in a physical value of the sensitive film.

9. The gas sensor according to claim 8, wherein a surface of the electrical insulation layer that contacts the sensitive film is flat to the degree that the maximum difference between the level of any low point and any high point in the surface is less than the thickness of the sensitive film.

10. The gas sensor according to claim 8, wherein the heater layer is frame-shaped, and a temperature control film for facilitating heat transfer from the heater layer is formed flatly inside the heater layer and on the same surface as the heater layer, wherein the outer periphery of the temperature control film is located between the inner periphery of the heater layer and the outer periphery of the sensitive film when the gas sensor is viewed in a plan view.

11. The gas sensor according to claims 8, wherein a corner of the heater layer is rounded.

12. The gas sensor according to claims 8, wherein the sensitive film is oval.

13. The gas sensor according to claim 8 further comprising a hollow cavity formed in the substrate below the heater layer and the sensitive film, wherein the hollow portion is spanned by the support film, and wherein tensile stress equal to or larger than 40 MPa and equal to or smaller than 150 MPa is applied to the support film.

14. The gas sensor according to claim 13, wherein the heater layer is located between an outer periphery of the hollow cavity and the outer periphery of the sensitive film when the gas sensor is viewed in a plan view.

15. The gas sensor according to claim 14, wherein the outer periphery of the hollow cavity at a surface of the substrate and the outer periphery of the sensitive film have similar shapes in a plan view.

16. The gas sensor according to claim 13, wherein a net tensile stress in the support film and all layers formed above the support film is equal to or larger than 40 MPa and equal to or smaller than 150 MPa.

17. The gas sensor according to claim 13, further comprising a projection formed on the support film, wherein the projection extends into the hollow cavity.

18. (canceled)

19. A method of fabricating a gas sensor comprising:
forming a heater layer such that the heater layer is supported by a substrate;
forming a first electrical insulation layer on the heater layer;
forming a detection electrode on the first electrical insulation layer;
forming a second electrical insulation layer on the first electrical insulation layer to cover the detection electrode;
flattening and thinning the second electrical insulation layer until a surface of the detection electrode is exposed; and
forming a sensitive film, a physical value of which changes when the sensitive film reacts to a gas being detected, on the flattened second electrical insulation layer to cover the exposed detection electrode; and
electrically connecting the detection electrode and the sensitive film.

20. The method of claim 19 further comprising:
forming a support film between the substrate and the heater layer;
forming a mask having an opening that corresponds generally to the location of the sensitive film, wherein the mask is formed on a face of the substrate that is opposite to the sensitive film; and
forming a hollow cavity in the substrate at a location that corresponds to the opening by etching the substrate through the mask.

21. The method of claim 20 further comprising forming a projection in the substrate such that the projection extends into the hollow cavity in the etching step, wherein the projection corresponds to an area covered by the mask.

22. The method of claim 20 further comprising:
forming a pad for the heater layer and a pad for the detection electrode; and
forming a filter for permitting a specific gas to reach the sensitive film.

23. A method of fabricating a gas sensor comprising:
simultaneously forming a heater layer and a detection electrode on a surface, wherein the thickness of the heater layer and that of the detection electrode differ;
covering the heater layer and the detection electrode with electrical insulation;
flattening and thinning the electrical insulation until a surface of the detection electrode is exposed; and
forming a sensitive film, a physical value of which changes when the sensitive film reacts to a gas being detected, on the flattened electrical insulation to cover the exposed detection electrode; and
electrically connecting the detection electrode and the sensitive film.

24. The method of claim 23, wherein the step of forming the heater layer and the detection electrode comprises:
forming a thin metal film, which provides material for the heater layer and the detection electrode;
forming a photoresist on the metal thin film;
exposing and developing the photoresist by using a photo mask having a fine pattern, the resolution of which is equal to or smaller than the resolution of the exposure, to form a pattern in which the thickness of an area that corresponds to the heater layer is less than the thickness of an area that corresponds to the detection electrode in the photoresist; and
etching the metal thin film by using the patterned photoresist such that the thickness of the heater layer is less than that of the detection electrode.

25. A method of fabricating a gas sensor comprising:
forming a heater layer such that the heater layer is supported by a substrate;
forming an electrical insulation layer facing the heater layer;
forming a sensitive film, a physical value of which varies when the sensitive film reacts to a gas being detected, on the electrical insulation layer such that the heater layer is located outside of the perimeter of the sensitive film when viewed in a plan view; and
forming a detection electrode for detecting changes in the physical value of the sensitive film on the sensitive film.
removing a part of the filter that corresponds to the pads after the hollow cavity is formed.

26-55. (canceled)
